(19) United States
(12) Patent Application Publication
(10) Pub. No.: US 2019/0390268 A1
(43) Pub. Date: Dec. 26, 2019

(54) CONTROLLED NANOPORE TRANSLOCATION UTILIZING EXTREMOPHILIC REPLICATION PROTEINS

(71) Applicant: ELECTRONIC BIOSCIENCES, INC., San Diego, CA (US)

(72) Inventors: Anna E.P. SCHIBEL, Snoqualmie, WA (US); Ryan Dunnam, San Diego, CA (US); Eric N. Ervin, Holladay, UT (US)

(21) Appl. No.: 16/452,355

(22) Filed: Jun. 25, 2019

Related U.S. Application Data
(60) Provisional application No. 62/690,182, filed on Jun. 26, 2018.

(51) Int. Cl. C12Q 1/6869; G01N 27/447

(52) U.S. Cl. CPC C12Q 1/6869; G01N 27/44791

(57) ABSTRACT

Devices and methods are provided for controlling translocation of single-stranded nucleic acid through a nanopore sensor or reader.

### RELATED PATENT APPLICATION(S)

This patent application claims the benefit of U.S. Provisional Patent Application No. 62/690,182 filed on Jun. 26, 2018 entitled “CONTROLLED NANOPORE TRANSLOCATION UTILIZING EXTREMOPHILIC REPLICATION PROTEINS,” naming Anna E. P. Schibel, Ryan Dunnam and Eric N. Ervin as inventors, and designated by attorney docket no. EBS-1010-PV. The entire content of the foregoing patent application is incorporated herein by reference, including all text, tables and drawings.

### FIELD

The technology relates in part to use of nanopore devices, such as for sequencing nucleic acids, for example.

### BACKGROUND

Since Church et al. first proposed the idea of polymer sequencing using a nanopore in 1995, nanopores have been extensively studied for their ability to directly sequence nucleic acids. These studies have proved to be extremely valuable with nanopore-based sequencing becoming a reality.

### SUMMARY

Provided herein in certain aspects are methods and devices for altering the translocation rate of nucleic acids through a nanopore as well as stretching or holding nucleic acids taught within a nanopore. Such methods and devices have nanopore-based DNA sequencing applications, for example Provided herein, in certain aspects is a method for translocating a single-stranded nucleic acid through a nanopore sensor or reader comprising contacting a single-stranded nucleic acid inserted in a nanopore sensor or reader with single-stranded binding proteins (SSBs) or replication protein A (RPAs) under binding conditions, thereby generating single-stranded nucleic acid with SSBs or RPAs bound to a first region of the single-stranded nucleic outside of the nanopore sensor or reader; and electrophoretically inducing translocation of a region of the single-stranded nucleic acid not bound by the SSBs or the RPAs through the nanopore sensor or reader.

Also provided in certain aspects is a method for translocating a single-stranded nucleic acid back and forth through a nanopore sensor or reader comprising contacting a single-stranded nucleic acid inserted in a nanopore sensor or reader with single-stranded binding proteins (SSBs) or replication protein A (RPAs) on the cis and trans sides of the nanopore sensor or reader under binding conditions; thereby generating single-stranded nucleic acid with SSBs or RPAs bound to a first region of the single-stranded nucleic on the cis side of the nanopore sensor or reader and single-stranded nucleic acid with SSBs or RPAs bound to a second region of the single-stranded nucleic on the trans side of the nanopore sensor or reader; and electrophoretically driving a third region of the single-stranded nucleic acid within the nanopore sensor or reader and not bound by the SSBs or the RPAs back and forth through the nanopore sensor or reader, whereby the third region of the single-stranded nucleic acid is translocated through the nanopore sensor or reader multiple times.

Also provided in certain aspects is a method to linearize ssDNA or ssRNA within a nanopore sensor or reader, comprising capturing ssDNA or ssRNA within a nanopore sensor or reader to produce captured ssDNA or ssRNA; contacting the captured ssDNA or ssRNA on the trans side of the nanopore sensor or reader with single-stranded binding proteins (SSBs) or replication protein A (RPAs) under binding conditions, wherein the SSBs or RPAs bind to a section of the ssDNA or ssRNA on the trans side to produce ssDNA or ssRNA with bound SSBs or bound RPAs; and moving the ssDNA or ssRNA back out of the nanopore sensor or reader, whereby the ssDNA or ssRNA is linearized.

Also provided in certain aspects is a method linearize ssDNA or ssRNA within a nanopore sensor or reader, comprising contacting ssDNA or ssRNA inserted in a nanopore sensor or reader comprising a cap, motor protein or enzyme bound to a first region of the ssDNA or ssRNA located on the cis side of the nanopore sensor or reader with single-stranded binding proteins (SSBs) or replication protein A (RPAs) on the trans side of the nanopore sensor or reader under binding conditions, thereby generating ssDNA or ssRNA with SSBs or RPAs bound to a second region of the ssDNA or ssRNA on the trans side of the nanopore sensor or reader; or contacting ssDNA or ssRNA inserted in a nanopore sensor or reader comprising a cap, motor protein or enzyme bound to a second region of ssDNA or ssRNA located on the trans side of the nanopore sensor or reader, with single-stranded binding proteins (SSBs) or replication protein A (RPAs) on the cis side of the nanopore sensor or reader under binding conditions; thereby generating ssDNA or ssRNA with SSBs or RPAs bound to a first region of the single-stranded nucleic on the cis side of the nanopore sensor or reader; and moving a third region of the ssDNA or ssRNA not bound by the SSBs, the RPAs, the cap, the motor protein or the enzyme through of the nanopore sensor or reader, whereby the ssDNA or ssRNA is linearized.

Also provided in certain aspects is a method for translocating ssDNA or ssRNA through a nanopore sensor or reader comprising contacting ssDNA or ssRNA inserted in a nanopore sensor or reader comprising a cap, motor protein or enzyme bound to a first region of the ssDNA or ssRNA located on the cis side of the nanopore sensor or reader with single-stranded binding proteins (SSBs) or replication protein A (RPAs) on the trans side of the nanopore sensor or reader under binding conditions; thereby generating ssDNA or ssRNA with SSBs or RPAs bound to a second region of the ssDNA or ssRNA on the trans side of the nanopore sensor or reader; or contacting ssDNA or ssRNA inserted in a nanopore sensor or reader comprising a cap, motor protein or enzyme bound to a second region of the ssDNA or ssRNA located on the trans side of the nanopore sensor or reader, with single-stranded binding proteins (SSBs) or replication protein A (RPAs) on the cis side of the nanopore sensor or reader under binding conditions, thereby generating ssDNA or ssRNA with SSBs or RPAs bound to a first region of the ssDNA or ssRNA on the cis side of the nanopore sensor or reader; and driving a third region of the ssDNA or ssRNA not bound by the SSBs, the RPAs, the cap, the motor protein or the enzyme through the nanopore sensor or reader, whereby the third region of the ssDNA or ssRNA is translocated through the nanopore sensor or reader.

Also provided in certain aspects is a method for preparing single-stranded DNA or single-stranded RNA for translocation through a nanopore sensor or reader, comprising separating the strands of double-stranded DNA or double-stranded RNA to produce single-stranded DNA or single-stranded RNA; contacting the single-stranded DNA or single-stranded RNA with binding proteins (SSBs) or replication protein A (RPAs) under binding conditions which the SSBs or RPAs bind to the single-stranded DNA or single-stranded RNA to produce single-stranded DNA or single-stranded RNA with bound SSBs or bound RPAs, and contacting the single-stranded DNA or single-stranded RNA with bound SSBs or bound RPAs with a nanopore sensor or reader.

Also provided in certain aspects is a method for translocating single-stranded DNA through a nanopore sensor or reader comprising contacting single-stranded DNA inserted in a nanopore sensor or reader with RPA3s from Haloferax volcanii under binding conditions comprising a salt concentration greater than 0.5M; thereby generating single-stranded DNA with RPA3s bound to a first region of the single-stranded DNA outside of the nanopore sensor or reader; and electrophoretically inducing translocation of a region of the single-stranded DNA not bound by the RPA3s through the nanopore sensor or reader.

Also provided in certain aspects is a method for translocating a single-stranded DNA through a biological nanopore sensor or reader comprising contacting single-stranded DNA inserted in a biological nanopore sensor or reader with RPA3s from Haloferax volcanii under binding conditions comprising a salt concentration between about 3.0M to 4.0M and a temperature less than or equal to 20° C., thereby generating single-stranded DNA with RPA3s bound to a first region of the single-stranded DNA outside of the nanopore sensor or reader; and electrophoretically inducing translocation of a region of the single-stranded DNA not bound by the RPA3s through the nanopore sensor or reader.

Also provided in certain aspects is a nanopore sensor or reader comprising a single-stranded nucleic acid, wherein a region of the single-stranded nucleic acid is on the cis side of a nanopore sensor or reader, a region of the single stranded nucleic acid is on the trans side of the nanopore sensor or reader and a region of the single-stranded nucleic acid is within the nanopore sensor or reader; the single-stranded nucleic acid comprises bound single-stranded binding proteins (SSBs) or replication protein A (RPAs) to a region on the cis side of the nanopore sensor or reader, to a region on the trans side of the nanopore sensor or reader or to a region on the cis side and a region on the trans side of the nanopore sensor or reader; and single-stranded binding proteins SSBs or RPAs are not bound to the single-stranded nucleic acid within the nanopore sensor or reader.

Also provided in certain aspects is a nanopore sensor or reader comprising a single-stranded nucleic acid, wherein a region of the single-stranded nucleic acid is on the cis side of a nanopore sensor or reader, a region of the single stranded nucleic acid is on the trans side of the nanopore sensor or reader and a region of the single-stranded nucleic acid is within the nanopore sensor or reader; the single-stranded nucleic acid comprises a cap, motor protein or enzyme bound to a region of the single-stranded nucleic acid located on the cis side of the nanopore sensor or reader and SSBs or RPAs bound to a region of the single-stranded nucleic on the trans side of the nanopore sensor or reader or the single-stranded nucleic acid comprises single-stranded binding proteins (SSBs) or replication protein A (RPAs) bound to a region on the cis side of the nanopore sensor or reader and a cap, motor protein or enzyme bound to a region of the single-stranded nucleic acid located on the trans side of the nanopore sensor or reader; and the SSBs or RPAs are not bound to the single-stranded nucleic acid within the nanopore sensor or reader.

Certain embodiments are described further in the following description, examples, claims and drawings.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIGS. 1A and 1B represent distributions of amplitude, standard deviation and duration of ACT-AGT-ACT translocation with and without 10:1 RPA3 additive.

FIG. 2 represent select ACT-AGT-ACT sequence event traces with and without 10:1 RPA3 additive.

FIGS. 3A and 3B show translocation time and blocking level distribution for free polyC100 (3) and HvRPA3 bound polyC100 (3B), translocating through wt-αHL.

FIG. 4 is a schematic of electrophoretically-induced ssDNA translocation through a nanopore reader under the binding influence of halophile-adapted RPA in high (>1 M) salt.

FIG. 5 shows a schematic of RPA bound to ssDNA on both cis and trans sides of the associated nanopore reader being electrophoretically driving back and forth through a nanopore reader (e.g.,—

FIG. 6 shows a schematic of monomeric RPA bound DNA translocation through a biological nanopore reader.

FIG. 7 shows a schematic of monomeric RPA bound DNA translocation through a synthetic nanopore.

FIG. 8 shows a schematic of monomeric RPA bound DNA translocation through a synthetic nanopore reader/sensor junction potential type device.

FIG. 9 shows a schematic of ssDNA with a cap or bound by an enzyme or motor on one side of a membrane, captured within a nanopore reader/sensor and bound by halophile and/or thermophile RPAs or SSBs on the opposite of the membrane.

FIG. 10 shows heterotrimeric RPA bound DNA translocation through a nanopore reader.

### DETAILED DESCRIPTION

Single-stranded DNA, and in some cases single-stranded RNA, can be bound with RPAs and/or SSBs and driven into and through any nanopore reader/sensor, synthetic or biological that is suitable for sequencing applications. Due to the SSB protein or RPA protein being too large to translocate through the nanopore, as the ssDNA or ssRNA traverses the nanopore, the SSB protein or RPA protein unbinds/unwinds from the single-stranded nucleic acid molecule. This unbinding process both slows down the ssDNA or ssRNA translocation speed relative to having no SSB or RPA present, in addition to linearizing or holding the ssDNA or ssRNA taught as it translocates, reducing the noise associated with freely translocating DNA or RNA through a nanopore as well as increasing the associated nucleotide resolution.

The technology described herein, exploits the capabilities of nucleic acid binding proteins (e.g., single-stranded binding proteins (SSBs) and replication protein A (RPAs)) from extremophiles that live in conditions such as extreme temperature, acidity, alkalinity, or chemical concentration to bind nucleic acids with high affinity under these conditions. The adapted cellular machinery from such extremophile organisms has significant application toward ion channel recording measurements (of both synthetic and biological pores) where temperature, pH, salt concentration, metal levels, etc. may be adjusted to influence the measurement sensitivity, molecular translocation rate, signal amplitude, signal noise, etc. A binding protein from an extremophile organism that binds DNA (and in some cases RNA) has specific application toward strand translocation experiments, where it can be utilized to, including but not limited to, prevent single stranded nucleic acid (e.g., ssDNA, ssRNA) crosslinking, minimize the formation of secondary structures and annealing events, stretch the strand against an applied driving force, and/or slow the associated nanopore translocation rate, etc. for improved single-to-noise ratio (SNR) and/or temporal resolution.

In some embodiments, a method is provided for slowing down the translocation speed of DNA through a nanopore as well as stretching or holding the DNA taught within the nanopore. Such methods have nanopore-based DNA sequencing applications. In essence, replication protein A (RPA) from extremophiles, or that live in conditions of extreme temperature, acidity, alkalinity, or chemical concentration, is mixed with single stranded DNA (ssDNA) at a high concentration of RPA to ssDNA. The RPA bound ssDNA molecule is then driven down into and through any nanopore reader, synthetic or biological, that is suitable for sequencing applications. Due to the RPA protein being too large to translocate through the nanopore, as the DNA traverses the nanopore, the RPA protein unbinds/unwinds from the DNA molecule. This unbinding process both slows down the ssDNA tranlocation speed relative to having no RPA present, in addition to linearizing or holding the DNA taught as it translocates, reducing the noise associated with freely translocating DNA through a nanopore as well as increasing the associated nucleotide resolution. Such method is ideally suited using experimental conditions in which RPAs from extremophiles have the highest binding affinity for ssDNA, i.e. high or low temperatures, high or low pH, and high chemical concentrations.

Single-Stranded Binding Proteins (SSBs)

Single-stranded binding proteins (SSBs) are non-specific DNA binding proteins in bacteria (including but not limited to Proteobacteria, Aquificae, Chlamydiae, Bacteroidetes, Chlorobi, Fibrobacteria, Spirochetes, Cyanobacteria, Chloroflexi, Deinococcus-Thermus, Thermotogae, Actinobacteria, Firmicutes, etc.), viruses (including but not limited to Caudovirales, Herpesvirales, Ligamenvirales, Mononegavirales, Nidovirales, Ortervirales, Picornavirales, Tymovirales, Bunyavirales), and eukaryotes (including but not limited to mitochondrial SSBs) that are involved in DNA replication, recombination, and repair.¹⁻² These proteins bind single-stranded DNA (ssDNA) with high affinity, protecting and stabilizing it while aiding the association of processive enzymes during DNA metabolism.

Replication Protein a (RPA)

Similarly, replication protein A (RPA) belongs to a class of proteins in eukaryotes (including but not limited to Animalia, Plantae, Fungi, Protista, etc.) and archaea (including but not limited Euryarchaeota such as Halobacteria, Methanomicrobia, Archaeoglobi, Thermoplasmata, Methanobacteria, Methanococci, Methanopyri, and Thermococci, etc., Nanoarchaeota, and Crenarchaeota such as Thermoproteales and Sulfolobales, etc.) that bind nonspecifically to ssDNA during cellular replication, recombination, and repair,³⁻⁴ and are a homolog to SSBs. The general function of these DNA-binding proteins is to protect ssDNA from secondary structure formation, annealing, damage, and/or modification of exposed bases (in its ssDNA form) by binding to the strand with high affinity during the cellular processes mentioned above.³⁻⁴

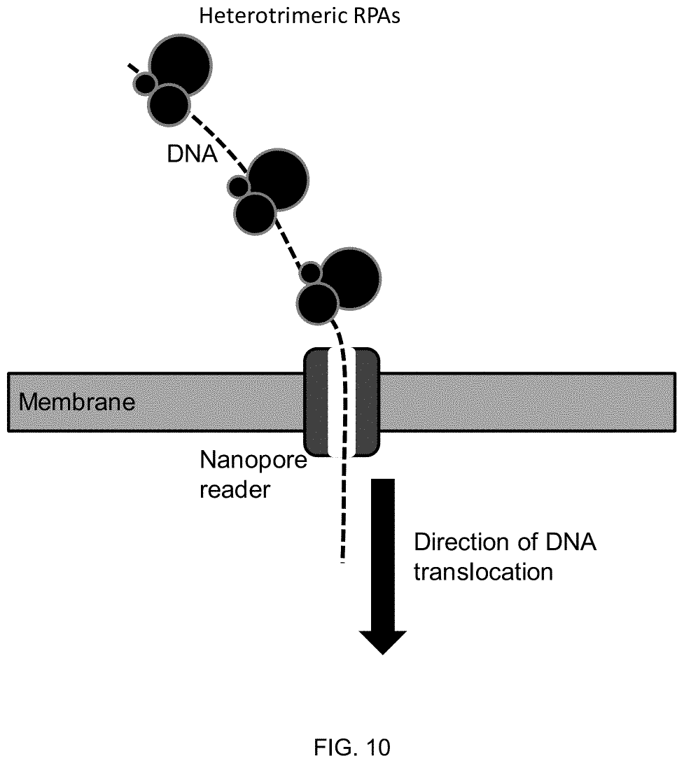

Organism can have multiple RPAs (e.g., RPA1, RPA2, RPA3, . . . , RPA14, . . . , RPA30, . . . , RPA70, etc.), and these RPAs may possess multiple subunits (e.g., including but not limited to a homodimer, homotrimer, homotetramer, heterodimer, heterotrimer, heterotetramer, etc.) or possess a single subunit (e.g., including but not limited to a monomeric protein, etc.) and function with variable complex organization, e.g., as a homomodimer, homotrimer, homotetramer, heterodimer, heterotrimer, heterotetramer, etc. Thus, this group of proteins, including SSBs, may have a wide range of sequences and ssDNA binding domains and differ in subunit composition and oligomerization or multimerization states,⁴⁻⁵ depending on the organism and environmental conditions (e.g., relative molecule concentrations, salt concentration, etc.). FIG. 10 shows a heterotrimeric RPA bound to ssDNA, for example.

Extremophile Binding Proteins

Various organisms have adapted to thrive under extreme environmental conditions, and these organisms are referred to as extremophiles. Extremophile organisms are organisms that survive and grow under extreme conditions, including but not limited to high temperatures, low temperatures, high pH, low pH, high salt concentrations, high pressure, low moisture, ionizing radiation, UV radiation, etc.⁶⁻⁷ To survive under these extreme conditions, extremophiles have adapted cellular components (nucleic acid binding proteins) including DNA replication, recombination, and repair machinery such as single-stranded binding proteins (SSBs) and replication protein A (RPAs).

In certain embodiments, extremophiles may include but are not limited to bacteria (e.g., Salinibacter, Thermus, Chryseobacterium, Cyanidium, Deinococcus, Salinicola, Halomonas, etc.), archaea (e.g., Ferroplasma, Haloarcula, Haloferax, Halogeometricum, Halococcus, Haloterrigena, Halorubrum, Halobacterium, Natronococcus, etc.), eukaryotes (e.g., Wallemia, Debaryomyces, Hortaea, etc.), and may be slight, moderate, and/or extreme. In some embodiments, extremophile organisms include but are not limited to thermophiles or hyperthermophiles (organisms that can survive and grow at temperatures at or above 45° C., e.g., Pyrolobus fumarii, Methanopyrus kandleri, etc.),⁶ psychrophiles or cryophiles (organisms that can survive and grow at temperatures at or below 15° C., e.g., Methanococcoides burtonii, Halorubrum lacusprofundi, etc.),⁶ alkaliphiles (organisms that can survive and grow at pH levels of 8.5 or above, e.g., Natronomonas pharaonis, Fusarium Bullatum, etc.),⁷⁻⁸ acidophiles (organisms that can survive and grow at pH levels of 3 or below, e.g., Picrophilus torridus, Ferroplasma acidiphilum, etc.),^{6, 9} and halophiles (organisms that can survive and grow at ˜2% or ˜0.34 M to ˜30% or ˜5.1 M salt, close to saturation conditions, e.g., Haloferax volcanii, Halobacterium salinarum, etc.),^{5-6, 10-11} etc. In certain embodiments, extremophiles may include but are not limited to metallotolerant organisms (organisms that can tolerate high levels of dissolved heavy metals), osmophiles (organisms that can survive and grow in high sugar concentrations), piezophiles or barophiles (organisms that can survive and grow under high pressure), radioresistant organisms (organisms that survive high levels of ionizing radiation), endoliths (organisms that can survive within rock or deep within the Earth's crust), xerophiles (organisms that can survive and grow under low moisture conditions), oligotrophs (organisms that can survive and grow in low nutrient environments) etc. In some embodiments, extremophile organisms (polyextremophiles) may be tolerant to a combination of extreme conditions (e.g., halophilic thermophiles, halophilic psychrophiles or cryophiles, halophilic alkaliphiles, halophilic acidophiles, etc.). For example, thermoacidophiles Galdieria sulphuraria tolerates high temperatures and acid conditions.¹²

Halophiles

In certain embodiments, SSBs and RPAs that bind to single-stranded nucleic acid of the described methods and nanopore sensors and readers are extremophiles that are halophiles. In some embodiments, the conditions for a halophile are a salt concentration of >0.3M, >0.5M, >1M, >1.5M, >2M, >2.5M, >3M, >3.5M, >4M, >4.5M, >5M, >5.5M or >6M.

Non-limiting examples of the types of electrolyte that could be used includes but is not limited to NaCl, LiCl, KCl, etc; or any salt with a cation consisting of ammonium, calcium, iron, magnesium, potassium, pyrdidinium, quanternary ammonium, sodium, or copper; or any salt with an anion consisting of acetate, carbonate, chloride, citrate, cyanide, fluoride, nitrate, nitrite, oxide, phosphate, or sulfate.

Non-limiting examples of the concentration of electrolyte that could be used included but is not limited to >0.3M, >0.5M, >1M, >1.5M, >2M, >2.5M, >3M, >3.5M, >4M, >4.5M, >5M, >5.5M, >6M, etc., which is also dependent on the solubility of the associated electrolyte.

In some embodiments, DNA binding proteins, include but are not limited to, DNA binding proteins from halophiles that function in the presence of various salt-forming ions, including but not limited to ammonium, calcium, iron, magnesium, potassium, sodium, copper, lithium, rubidium, cesium, fluoride, chloride, acetate, nitrate, phosphate, phosphate, sulfate, etc.

In certain embodiments DNA binding proteins include RPAs or SSBs from a halophilic organism (i.e., an organism that can grow in salt conditions above 0.2 M), including but not limited to those organisms that are halotolerant (in approximately 1-6% salt), moderate halophiles (in approximately 6-15% salt), and extreme halophiles (in approximately 15-30% salt)^{13,14}, as RPAs or SSBs from these organisms are likely to be able to bind ssDNA with high affinity under high (>0.2 M) salt conditions. In some embodiments, DNA binding proteins can include, but are not limited to, DNA binding proteins from any halophiles belonging to Halobacterium (e.g., Halobacterium salinarum, Halobacterium noricense, etc.), Haloarcula (e.g., Haloarcula vallismortis, Haloarcula marismortui, Haloarcula hispanica, Haloarcula japonica, Haloarcula argentinensis, Haloarcula quadrata, etc.), Halobaculum (e.g., Halobaculum gomorrense, etc.), Halococcus (e.g., Halococcus morrhuae, Halococcus saccharolyticus, Halococcus salifodinae, Halococcus dombrowskii, etc.), Haloferax (e.g., Haloferax volcanii, Haloferax gibbonsii, Haloferax denitrificans, Haloferax mediterranei, Haloferax alexandrines, Haloferax lucentensis, Haloferax sulfurifontis, Haloferax elongans, etc.), Halogeometricum (e.g., Halogeometricum boringuense, etc.), Halorhabdus (e.g., Halorhabdus utahensis, etc.), Halorubrum (e.g., Halorubrum saccharovorum, Halorubrum sodomense, Halorubrum lacusprofundi, Halorubrum coriense, Halorubrum distributum, Halorubrum kocurii, Halorubrum vacuolatum, Halorubrum trapanicum, Halorubrum tebenquichense, Halorubrum terrestre, Halorubrum xinjiangense, Halorubrum alkaliphilum, etc.), Haloterrigena (e.g., Haloterrigena turkmenica, Haloterrigena thermotolerans, etc.), Natrialba (e.g., Natrialba asiatica, Natrialba magadii, Natrialba taiwanensis, Natrialba aegyptiaca, Natrialba hulunbeire14nsis, Natrialba chachannaoensis, etc.), Natrinema (e.g., Natrinema pellirubrum, Natrinema pallidum, Natrinema versiforme, Natrinema altunense, etc.), Natronobacterium (e.g., Natronobacterium gregoryi, etc.), Natronococcus (e.g., Natronococcus occultus, Natronococcus amylolyticus, etc.), Natronomonas (e.g., Natronomonas pharaonis, etc.), Natronorubrum (e.g., Natronorubrum bangense, Natronorubrum tibetense, etc.), Halomicrobium (e.g., Halomicrobium mukohataei, etc.), Halobiforma (e.g., Halobiforma haloterrestris, Halobiforma nitratireducens, Halobiforma lacisalsi, etc.), Halosimplex (e.g., Halosimplex carlsbadense, etc.), Halalkalicoccus (e.g., Halalkalicoccus tibetensis, etc.), Halovivax (e.g., Halovivax asiaticus, etc.),

In certain embodiments, DNA binding proteins, include but are not limited to, DNA binding proteins from halophiles that belong to bacterial genera Bacillus, Halomonas, Pseudomonas, Micrococcus, Alcaligenes, Staphylococcus, Actinomycetes, Corynebacterium, Marinobacter, Planococcus, Arthrobacter and Nocardia, etc.

In some embodiments, DNA binding proteins, include but are not limited to, DNA binding proteins from Halorhodospira halophila, Marinobacter hydrocarbonoclasticus, Marinobacter hydrocarbonoclasticus, Halomonas elongata, Deleya halophila, Desulfovibrio halophilus, Desulfohalobium retbaense, Flavobacterium halmephilum, Haloanaerobacter chitinovorans, Haloanaerobium praevalens, Halobacteroides halobius, Halomonas elongate, Halomonas eurihalina, Halomonas halodenitrificans, Halomonas halodurans, Halomonas subglaciescola, Paracoccus halodenitrificans, Pseudomonas beijerinckii, Pseudomonas halophila, Spirochaeta halophila, Sporohalobacter lortetii, Sporohalobacter marismortui, Vibrio costicola, Marinococcus albus, Marinococcus halobius, Sporosarcina halophila, Ectothiorhodospira vacuolata, Rhodospirillum salexigens, and Rhodospirillum salinarum, etc.

In some embodiments, a DNA-binding protein is from Haloferax volcanii. In certain embodiments, a DNA-binding protein is an RPA from Haloferax volcanii. In some embodiments, the RPA from Haloferax volcanii is RPA3. RPA3 from halophile Haloferax volcanii, HvRPA3. HvRPA3, the smallest of the three H. volcanii RPAs, is a monomeric protein that has been demonstrated to be capable of binding nucleotides of ssDNA with high affinity in salt concentrations of at least up to 3 M.⁵ Some adaptations to enable organism survival and growth under extreme conditions may include but are not limited to increased disulfide bonds, increased salt-bridging, increased surface charges, increased acidic residues, decreased hydrophobic residues, etc.⁷

In some embodiments, the binding conditions for RPA3 binding single-stranded nucleic acid comprises a salt concentration between 3M and 4M. In some embodiments, the temperature for RPA3 binding is less than about 32° C., less than or equal to about 20° C. or about 5° C.

Thermophiles

In certain embodiments, SSBs and RPAs of the described methods and nanopore sensors and readers are extremophiles that are thermophiles.

Non-limiting examples of the temperature range that could be used includes but is not limited to 00° C. to 100° C., above 32° C., below 32° C., below 10° C., below 5° C., below 00° C., below −5° C., etc.

For clarification generally a thermophile functions at a high temperature of greater than 32° C. or a low temperature of less than 5° C. In some embodiments, the binding conditions comprise high temperature and the temperature is above 32° C. or the binding conditions comprise low temperature and the temperature is below 5° C., below 00° C. or below −5° C.

In some embodiments, a DNA-binding protein is TaqSSB from Thermus aquaticus.

In certain embodiments, the binding proteins described herein, including SSBs and RPAs, are native proteins or a portion thereof. In certain embodiments, the binding proteins described herein, including SSBs and RPAs, are recombinant proteins. In certain embodiments, the binding proteins described herein, including SSBs and RPAs, are mutated, engineered, chemically modified, or is a mutant form.

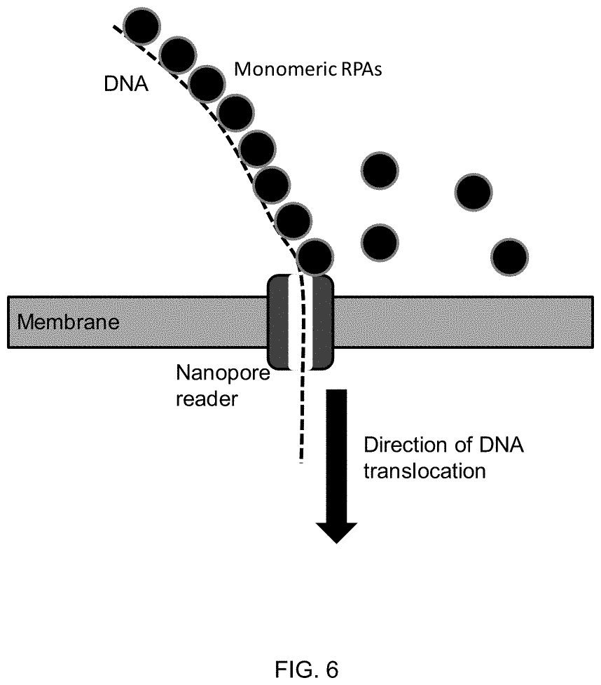

In certain embodiments, the described SSBs or RPAs comprise one or more subunits that are in one or more oligomerization or multimerization states. In certain embodiments, the described SSBs or RPAs comprise single subunits or monomeric proteins (e.g., see FIGS. 6, 7 and 8). For example, RPA3 of Haloferax volcanii. In certain embodiments, the described SSBs or RPAs comprise multiple subunits and are homodimers, homotrimers, homotetramers, heterodimers, heterotrimers or heterotetramers.

Nucleic Acids

In certain embodiments, a single-stranded nucleic acid is DNA, RNA or cDNA. In some embodiments, a single-stranded nucleic acid is prepared from a double-stranded nucleic acid or a single-stranded nucleic acid that has formed double-stranded regions by folding or hybridizing with itself. In certain embodiments, single-stranded DNA or single-stranded RNA is prepared for insertion into a nanopore sensor or reader by separating the strands of DNA or RNA to produce single-stranded DNA (ssDNA) or single-stranded RNA (ssRNA). In some embodiments, strand separation if followed by binding of SSBs or RPAs. In some embodiments, single-stranded DNA or single-stranded RNA is inhibited from hybridizing with itself or folding onto itself by contact with SSBs or RPAs. In some embodiments, separating strands is by chemical denaturation. In some embodiments, chemical denaturation uses NaOH.

Nanopore Sensors or Readers

In some embodiments, a nanopore sensor or reader comprises a nanopore provided in a device or apparatus that allows for sensing of a nucleic acid that pass through the nanopore channel. In certain embodiments, the apparatus further comprises a DC measurement system. In some embodiments, the apparatus further comprises an AC measurement system. In certain embodiments, the apparatus further comprises an AC/DC measurement system.

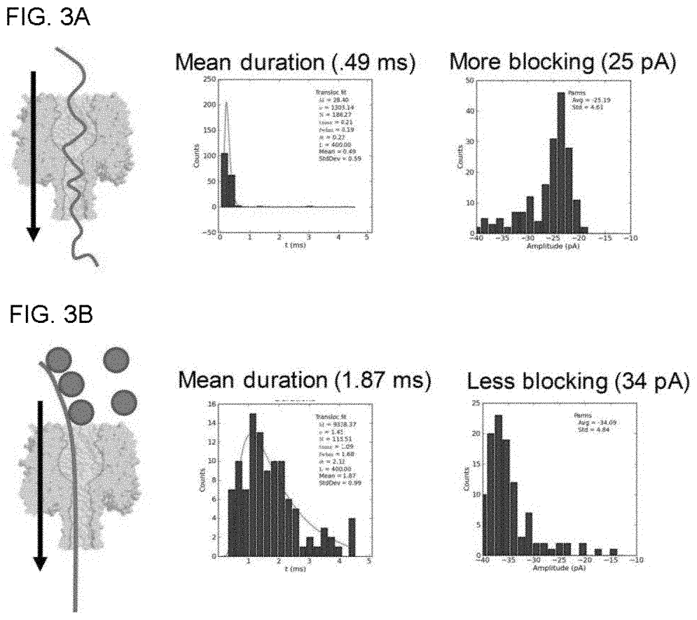

In certain embodiments, the nanopore sensor or reader is a biological nanopore sensor or reader (e.g., see FIGS. 3B, 5 and 6). In some embodiments, the biological nanopore sensor or reader is alpha-hemolysin (αHL), aerolysin, Mycobacterium smegmatis porin A (MspA), Escherichia coli CsgG, or outer membrane protein F (OmpF).

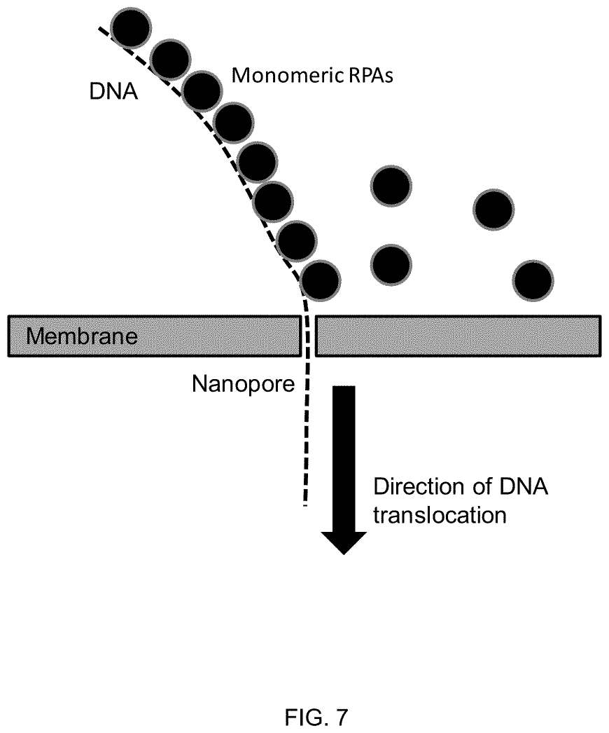

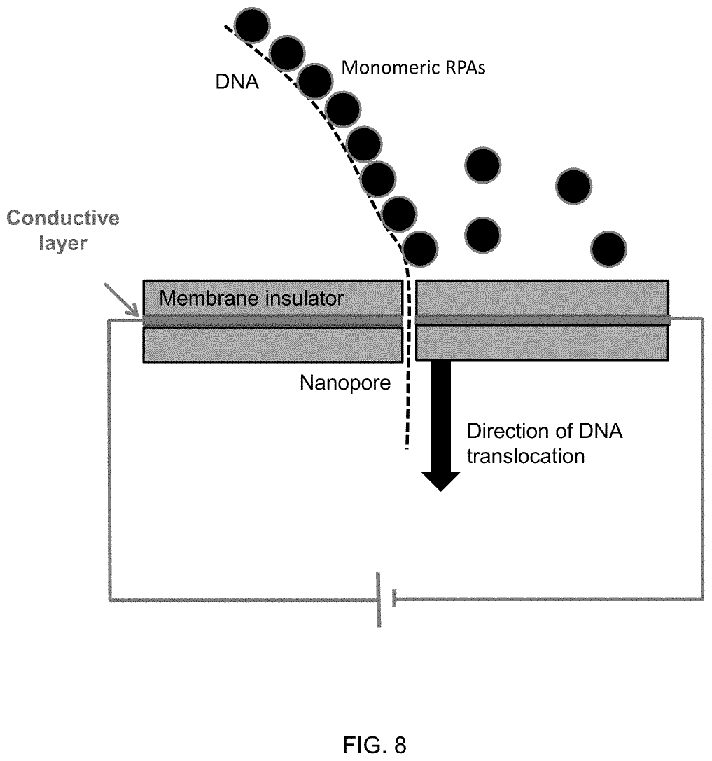

In certain embodiments, the nanopore sensor or reader is a synthetic or solid-state nanopore sensor or reader (e.g., see FIGS. 7 and 8). In some embodiments, the synthetic nanopore sensor or reader comprises an aperture with a diameter that prevents the single-stranded binding proteins (SSBs) or replication protein A (RPAs) bound to single-stranded nucleic acid from entering the nanopore sensor or reader. In some embodiments, the diameter is about 0.2 nanometers to about 10 nanometers, or about 0.20 nanometers, about 0.25 nanometers, about 0.5 nanometers, about 1 nanometer, about 1.5 nanometers, about 2 nanometers, about 2.5 nanometers, about 3 nanometers, about 3.5 nanometers, about 4 nanometers, about 4.5 nanometers, about 5 nanometers, about 6 nanometers, about 7 nanometers, about 8 nanometers, about 9 nanometers or about 10 nanometers.

Conditions

Nucleic acid binding proteins (e.g., SSBs and RPAs) bind to single-stranded nucleic acid (e.g., ssDNA, ssRNA) under specific conditions or binding conditions. Typically the conditions or binding conditions for a nucleic acid binding protein are conditions that enable high affinity binding to the nucleic acid. In certain embodiments, the methods and devices described herein, utilize the conditions which SSB's or RPA's from extremophile organisms bind to ssDNA or ssRNA. These conditions enable high affinity binding to ssDNA or ssRNA and allow for adjustment of properties associated with the translocation of ssDNA or ssRNA through nanopore sensors or readers.

For example, the point of the associated method is to utilize the evolutionary imparted capabilities of the halophile RPAs or SSBs or thermophiles RPAs or SSBs, to bind to ssDNA and in some cases ssRNA, with high affinity under high salt and/or extreme temperature conditions.

In certain embodiments, the SSBs or RPAs of the methods and nanopore sensors and readers described herein are from an extremophile. In some embodiments, the conditions under which SSBs or RPAs from an extremophile bind single-stranded nucleic acid comprise conditions that are similar to the conditions of the environment in which an extremophile is found in nature. In certain embodiments, the conditions under which the he SSBs or RPAs of the described methods and nanopore sensors and readers bind to single-stranded nucleic acid are the conditions under which SSBs and RPAs exhibit the highest binding affinity for single-stranded nucleic acid. In some embodiments, the conditions comprise high temperature, low temperature, high pH, low pH, high salt concentration, high metal concentration, high chemical concentration or combinations thereof. In some embodiments, the conditions comprise high salt and/or temperature less than or equal to 10° C. In some embodiments, the conditions comprise high salt concentration and temperature less than or equal to 20° C.

In some embodiments, binding conditions comprise contacting a single-stranded nucleic acid with SSBs or RPAs prior to insertion of the single-stranded nucleic acid into a nanopore sensor or reader. In some embodiments, binding conditions comprise contacting a single-stranded nucleic acid that is inserted into a nanopore sensor or reader with SSBs or RPAs.

In certain embodiments, a single-stranded nucleic acid inserted into a nanopore sensor or reader has a portion of the molecule outside of and on the cis side of the nanopore sensor or reader and a portion of the molecule within the nanopore sensor or reader. In certain embodiments, a single-stranded nucleic acid inserted into a nanopore sensor or reader has a portion of the molecule outside of and on the trans side of the nanopore sensor or reader and a portion of the molecule within the nanopore sensor or reader. In certain embodiments, a single-stranded nucleic acid inserted into a nanopore sensor or reader has a portion of the molecule outside of and on the cis side of the nanopore sensor or reader, a portion of the molecule outside of and on the trans side of the nanopore sensor or reader and a portion of the molecule within the nanopore sensor or reader.

In some embodiments, a portion of a single-stranded nucleic acid outside of and on the cis side of a nanopore sensor or reader (bulk solution side) comprises a first region of the single-stranded nucleic acid. In some embodiments, a portion of a single-stranded nucleic acid outside of and on the trans side of a nanopore sensor or reader comprises a second region of the single-stranded nucleic acid. In some embodiments, a portion of a single-stranded nucleic acid within a nanopore sensor or reader does not have bound SSBs or RPAS and comprises a third region of the single-stranded nucleic acid.

In certain embodiments, SSBs or RPAs are contacted with single-stranded nucleic acid at a high concentration of SSBs or RPAs to single-stranded nucleic acid. In some embodiments, the concentration of SSBs or RPAs to single-stranded nucleic acid is greater than or equal to about 10:1, greater than or equal to about 100:1, or > about 10:1, > about 20:1, > about 30:1, > about 40:1, > about 50:1, > about 60:1, > about 70:1, > about 80:1, > about 90:1 or > about 100:1. In certain embodiments, either SSBs or RPEs of a single species are bound to single-stranded nucleic acid. In some embodiments, conditions influence the binding of the species of SSBs or RPAs. In certain embodiments, either SSBs or RPEs of more than one species of SSBs or RPAs are bound to single-stranded nucleic acid. In some embodiments, conditions influence the binding of the more than one species of SSBs or RPAs. In certain embodiments, both SSBs and RPAs can be bound to a single-stranded nucleic acid. In some embodiments, conditions influence the binding of both SSBs and RPAs.

Cap, Enzyme or Motor Protein

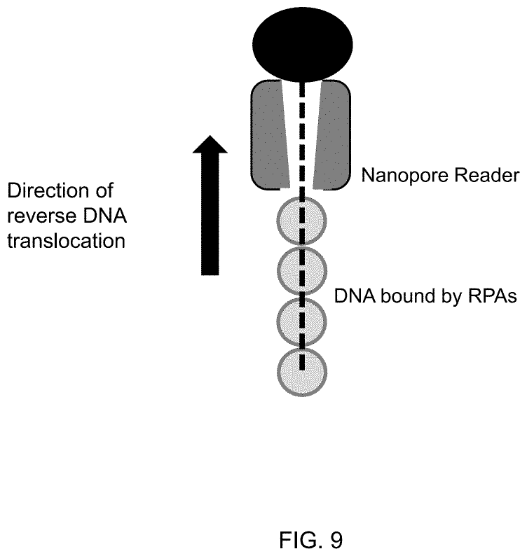

In certain embodiments, a single-stranded nucleic comprises a cap. In some embodiments, the single-stranded nucleic acid is DNA. In certain embodiments, the single-stranded nucleic is RNA. In some embodiments, the cap is located at the end or terminus of the strand (5′ end or 3′ end). In some embodiments, a cap is located along the length of a stand, but not at the terminus of the strand. In some embodiments, the single-stranded nucleic acid comprising a cap is inserted into a nanopore sensor or reader with the cap located on a portion of the single-stranded nucleic acid on the cis side of the nanopore sensor or reader and bound to a first region of the single-stranded nucleic acid. In some embodiments, the single-stranded nucleic acid comprising a cap is inserted into a nanopore sensor or reader with the cap located on a portion of the single-stranded nucleic acid on the trans side of the nanopore sensor or reader and bound to a second region of the single-stranded nucleic acid. In certain embodiments, a cap comprises a molecule that bound to the single-stranded nucleic acid will not fit through a pore of a nanopore sensor or reader and once bound to a single-stranded nucleic acid remains bound to the nucleic acid when subjected to forces generated when single-stranded nucleic acid is translocated through a nanopore sensor or reader. In some embodiments, an attached cap can act as a stop for the translocation of single-stranded nucleic acid through a nanopore sensor or reader. In some embodiments, a cap determines the direction of translocation, based on whether it is bound on the 5′ side or 3′ side of the single-stranded nucleic acid relative to the nanopore. In some embodiments, a cap can bind adjacent to a specific section of a single-stranded nucleic acid that is to be sequenced and act to target the region for sequencing. In some embodiments, a cap can be an adduct. In some embodiments a cap can be a large bulky protein that binds to nucleic acid and cannot be removed. In some embodiments, a cap is biotin/streptavidin, a hairpin or a g-quadreplex protein. In certain embodiments, ssDNA comprising a cap is captured or trapped within a nanopore on one side of a nanopore (cis or trans). The ssDNA is contacted with SSBs or RPAs on the opposite side of the nanopore (trans or cis) and the SSBs or RPAs become bound to the ssDNA. The single-stranded molecule is then electrophoretically driven through the nanopore in the direction of the cap, such that the single-stranded nucleic acid is held taught, slowed, stretched and/or linearized. FIG. 9 illustrates a cap bound to ssDNA on the cis side of a nanopore reader and RPAs bound to ssDNA on the trans side and translocation of the ssDNA out of the nanopore reader (black oval represents a cap).

In certain embodiments, a single-stranded nucleic comprises a bound enzyme or motor protein. In some embodiments, the single-stranded nucleic acid is DNA. In certain embodiments, the single-stranded nucleic is RNA. In some embodiments, an enzyme or motor protein is located at the end or terminus of the strand (5′ end or 3′ end). In some embodiments, an enzyme or motor protein is located along the length of a stand, but not at the terminus of the strand. In some embodiments, the single-stranded nucleic acid comprising an enzyme or motor protein is inserted into a nanopore sensor or reader with the enzyme or motor protein located on a portion of the single-stranded nucleic acid on the cis side of the nanopore sensor or reader and bound to a first region of the single-stranded nucleic acid. In some embodiments, the single-stranded nucleic acid comprising a an enzyme or motor protein is inserted into a nanopore sensor or reader with the enzyme or motor protein located on a portion of the single-stranded nucleic acid on the trans side of the nanopore sensor or reader and bound to a second region of the single-stranded nucleic acid. In certain embodiments, an enzyme or motor protein moves single-stranded nucleic acid (e.g., ssDNA or ssRNA) through a nanopore sensor or reader. In certain embodiments, an enzyme or motor protein is a polymerase, a helicase, a topoisomerase or a gyrase. In some embodiments, an enzyme or motor protein is from an extremophile, a halophile or a thermophile. In some embodiments, an enzyme or a motor protein moves or ratchets ssDNA through a nanopore sensor or reader. In certain embodiments, an enzyme or motor protein bound to ssDNA is initially captured within a nanopore and then the ssDNA is bound by RPAs on the opposite side to which the enzyme or motor protein is bound. The ssDNA is then driven back out of the nanopore against the bound RPAs via the enzyme/motor protein (e.g., see FIG. 9, black oval represents an enzyme or motor protein), such that the ssDNA is held taught, slowed, stretched, and/or linearized.

In some embodiments, an enzyme or motor protein enzyme functions at high salt concentrations and/or high or low temperatures. In some embodiments, the directionality of moving ssDNA or ssRNA through a nanopore sensor or reader is determined by whether the enzyme or motor protein is bound to the ssDNA or ssRNA inserted into a nanopore sensor or reader on the 3′ or 5′ side of the molecule. If an enzyme or motor protein requires any other substrates or reagents to function, these can be supplied either attached or in bulk solution.

Translocation

Translocation through a nanopore sensor or reader of single-stranded nucleic acid having bound SSBs or RPAs has many advantages over translocation of single-stranded nucleic acid without bound SSBs or RPAs. Without being held to a theory, bound SSBs or RPAs result in single-stranded nucleic acid being stretched or linearized during translocation as opposed to compressed, squiggly, folded, twisted as shown in FIGS. 3A and 3B. The bound SSBs or RPAs are “pulled against” as they contact the opening aperture of a nanopore and are physically forced off the single-stranded nucleic acid. This allows both the single-stranded nucleic acid to be stretched/linearized during translocation, as well as slowing down the translocation rate (i.e., translocation event duration). A slower rate of translocation allows the use of a lower measurement bandwidth, reducing the noise of associated measurements and improving sequencing capabilities. A single-stranded nucleic acid that is stretched/linearized improves inter-nucleotide resolution and thus higher sequence resolution. Single-stranded nucleic acid with bound SSBs or RPAs that is stretched or linearized as it is translocated through a nanopore sensor or reader exhibits a higher blocked/translocation current level relative to the blocked/translocation current level of single-stranded nucleic acid without bound SSBs or RPAs.

In certain embodiments, translocation of a third region of a single-stranded nucleic acid inserted in the nanopore sensor or reader which is not bound by SSBs or RPAs, but having SSBs or RPAs bound to a first region of the single stranded nucleic acid, having SSBs or RPAs bound to a second region of the single-stranded nucleic acid or having SSBs or RPAs bound to a first region and a second region of the single-stranded nucleic acid is slower relative to translocation of a third region of a single-stranded nucleic acid inserted in the nanopore sensor or reader which is not bound by SSBs or RPAs and not having SSBs or RPAs bound to a first region of the single stranded nucleic acid, not having SSBs or RPAs bound to a second region of the single-stranded nucleic acid or not having SSBs or RPAs bound to a first region and a second region of the single-stranded nucleic acid.

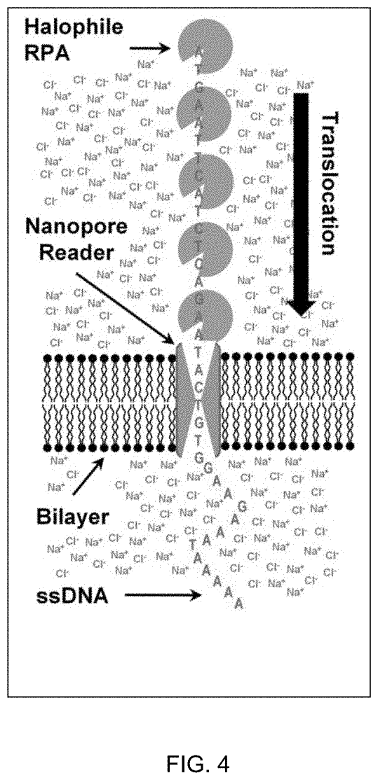

FIG. 4 illustrates ssDNA with halophile-adapted RPA bound to the ssDNA on the cis side of a nanopore reader (i.e., first region of the nucleic acid). Translocation of the ssDNA is electrophoretically induced in high salt conditions. The region of the ssDNA not bound by RPAs (third region) moves through the nanopore reader. FIGS. 6, 7 and 8 illustrate translocation through a nanopore sensors or readers of ssDNA with RPAs bound to the ssDNA on the cis side of a nanopore reader (i.e., first region of the nucleic acid). As the ssDNA is translocated through the nanopore sensor or reader by an applied electric current or by the action of a bound enzyme or motor protein RPAs are forced off or stripped from the ssDNA as they contact an aperture of the nanopore which they cannot fit through. It is apparent that the third region of the ssDNA that is moving through the nanopore sensor or reader is changing and is generated as RPAs are stripped of a first region and/or a second region of the ssDNA to which they were bound. As SSBs or RPAs are stripped from the first and/or second region of the single-stranded nucleic acid, the region no longer having bound SSBs or RPAs can now enter the nanopore sensor or reader and becomes a new third region that moves through the nanopore sensor or reader.

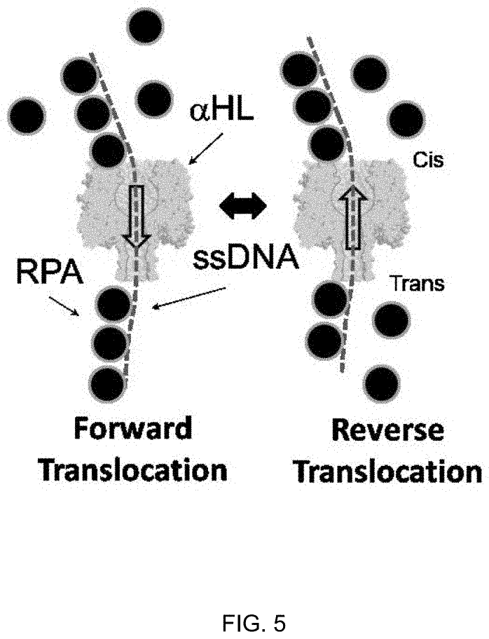

FIG. 5 illustrates RPAs bound to ssDNA on both the cis and trans side of a nanopore reader (i.e., the first region DNA and the second region of the ssDNA). The third region of the ssDNA, not having bound RPAs is within the nanopore reader. Translocation is sequentially switched between forward and reverse directions.

In some embodiments, translocation of a third region of a single-stranded nucleic acid inserted in the nanopore sensor or reader which is not bound by SSBs or RPAs, but having SSBs or RPAs bound to a first region of the single stranded nucleic acid, having SSBs or RPAs bound to a second region of the single-stranded nucleic acid or having SSBs or RPAs bound to a first region and a second region of the single-stranded nucleic acid is at a rate of about 100 microseconds to about 10 milliseconds, or about 100 microseconds, about 200 microseconds, about 300 microseconds, about 400 microseconds, about 500 microseconds, about 600 microseconds, about 700 microseconds, about 800 microseconds, about 900 microseconds, about 1 milliseconds, about 2 milliseconds, about 3 milliseconds, about 4 milliseconds, about 5 milliseconds, about 6 milliseconds, about 7 milliseconds, about 8 milliseconds, about 9 milliseconds or about 10 milliseconds.

In certain embodiments, DC bias (i.e., DC driving voltage) is used to electrophoretically control translocation of the single-stranded nucleic acid through the pore of a nanopore sensor or reader. In some embodiments, bound single-stranded binding proteins (e.g., SSBs or RPAs) enable the use of higher DC driving voltages, then in the absence of SSBs or RPAs, to monitor translocation of single-stranded nucleic acid through a nanopore sensor or reader. A higher driving voltage increases the electrophoretic force placed on negatively charged DNA molecules within a pore and results in more stretch/linearization when opposed by bound SSBs or RPAs. In certain embodiments, the DC bias is in the range of about 1 mV to about 300 mV or greater (e.g. 1 mV, 2 mV, 3, mV, 4 mV, 5 mV, 6 mV, 7 mV, 8 mV, 9 mV, 10 mV, 15, mV, 20 mV, 25 mV, 30 mV, 35 mV, 40 mV, 45 mV, 50 mV, 60 mV, 70 mV, 80 mV, 90 mV, 100 mV, 110 mV, 120 mV, 130 mV, 140 mV, 150 mV, 160 mV, 170 mV, 180 mV, 190 mV, 200 mV, 210 mV, 220 mV, 230 mV, 240 mV, 250 mV, 260 mV, 270 mV, 280 mV, 290 mV or 300 mV). In some embodiments, the DC driving voltages can be up to about −250 mV.

In some embodiments, a single-stranded nucleic acid is translocated through a nanopore sensor or reader by a bound enzyme or motor protein. In some embodiments, an enzyme or motor protein is bound to single-stranded nucleic acid inserted into a nanopore sensor or reader on the cis side of the nanopore. In some embodiments, an enzyme or motor protein is bound to single-stranded nucleic acid inserted into a nanopore sensor or reader on the trans side of the nanopore.

In some embodiments, the effect of SSBs or RPAs on the translocation rate of single-stranded nucleic acid through a nanopore sensor or reader is sequence independent. In some embodiments, the effect of SSBs or RPAs on the translocation rate of single-stranded nucleic acid through a nanopore sensor or reader is sequence dependent.

While the ability of a DNA binding protein to binding DNA under high salt conditions is one feature that may be utilized during DNA translocation experiments to influence the translocation rate and/or signal, the ability to bind DNA under various other conditions may also be imparted to DNA binding proteins from alternative extremophile organisms,

Linearizing Single-Stranded Nucleic Acid

In certain embodiments, single-stranded nucleic acid with bound SSBs or RPAs is linearized as the molecules translocates through the nanopore sensor or reader. In some embodiments, a linearized single-stranded nucleic acid results in less blocking of current in the nanopore and accordingly a higher current (e.g., see FIG. 3B). FIGS. 6, 7 and 8 shows linearization of ssDNA with bound monomeric RPAs as the molecule translocates through a nanopore and FIG. 10 shows linearization of ssDNA with bound heterotrimeric RPAs as the molecule translocates through a nanopore. In some embodiments, translocation of single-stranded nucleic acid with a cap, enzyme or motor protein bound to a section of the molecule on one side of a nanopore and SSBs and/or RPAs bound to the molecule on the other side of a nanopore translocated through a nanopore sensor or reader results in linearized single-stranded nucleic acid (e.g., see FIG. 9).

Recording Measurements

When utilizing extremophile RPAs or SSBs to control or aid in the translocation through a nanopore, synthetic or biological, a DC bias can be used to monitor the conductance of the pore and electrophoretic ally control the translocation of the bound DNA molecule through the pore; an AC bias can be used to monitor the conductance of the pore while an accompanying DC bias is used to electrophoretically control the translocation of the bound DNA through the pore; or a motor or enzyme that functions in high salt or at high or low temperatures could be used to control the translocation of the bound DNA molecule through the pore, while an AC or DC bias is used to monitor the conductance of the nanopore and thus determine the sequence of the DNA strand as it was translocates based on the accompanying current as a function of time signature.

FIG. 8 illustrates an example of a junction potential type devices that measures current through each individual nucleotide as they pass through the electrode or conductor junction or gap. Other nanopore sensors or readers that can detect nucleotides as they pass through the nanopores channel can be used for sequencing in the methods described herein.

In certain embodiments, conditions are adjusted to influence recording measurements of a nanopore sensor or reader. For example, conditions can be adjusted to obtain more useful target event durations and/or signal to noise ratios. Conditions can be any condition as previously described (e.g., salt concentration, temperature) that affects the binding of single-stranded binding proteins (e.g., SSBs or RPAs) to single-stranded nucleic acid and accordingly alters the rate of translocation and/or the linearity (degree of stretching) of the single-stranded nucleic acid. Conditions, as used herein, can also be conditions that effects the rate of translocation independent of single-stranded binding proteins (e.g., temperature when the SSBs or RPAs are from a halophile that is not also a thermophile). In some embodiments, the conditions comprise temperature and/or salt concentration.

In some embodiments, the recording measurements are current as a function of time. In some embodiments, the current as a function of time noise level is reduced by utilizing bound SSBs or RPAs. In some embodiments, the recording measurements are sensitivity, translocation time, signal amplitude, signal noise, signal to noise ratio and/or temporal resolution. In some embodiments, the recording measurements comprise sequence dependent current signatures. In some embodiments, the recording measurements in the presence of SSBs or RPAs bound to a first region of a single-stranded nucleic acid, bound to a second region of a single-stranded nucleic acid or bound to a first and a second region of a single-stranded nucleic acid comprise a lower bandwidth measurement relative to the bandwidth measurement for recording measurements in the absence of SSBs or RPAs bound to a first region of the single-stranded nucleic acid, bound to a second region of a single-stranded nucleic acid or bound to a first and a second region of a single-stranded nucleic acid. Bandwidth or frequency range can have a lower bandwidth measurement as translocation rate is slowed due to binding of SSBs or RPAs and thus the noise level is reduced.

In certain embodiments, methods and nanopore sensors described herein comprise a plurality of nanopore sensors and readers, each of which can translocate a molecule of the single-stranded nucleic acid. In some embodiments, the recording measurements are multiplexed through multiple nanopore sensors or readers. The utilization of a multiplexed platform, in which multiple nanopore sensors or readers are utilized simultaneously, will enable relatively high throughput and reasonable sample characterization times.

Sequencing

In certain embodiments, the methods and nanopore sensors or reader described herein are used in a sequencing process. Either a biological naopore or a solid state nanome (synthetic nanopore) can be utilized for sequencing. In some embodiments, the sequence of a single-stranded nucleic acid or a portion thereof is determined. In some embodiments, determining the sequence of the single-stranded nucleic acid or a portion thereof with SSBs or RPAs bound to a first region of the single-stranded nucleic acid increases the inter-nucleotide resolution relative to the inter-nucleotide resolution for determining the sequence of the single-stranded nucleic acid without SSBs or RPAs bound to a first region of the single-stranded nucleic acid.

The utilization of extremophile RPAs or SSBs to hold onto ssDNA and in some instances ssRNA as it is driven through a nanopore reader or sensor, helps to linearize or stretch the DNA or RNA as it translocates, holding it taught, arranging the individual nucleotides on the strand in single file order, as well as potentially increasing the inter-nucleotide distance between each associated nucleotide or base that makes up the DNA or RNA strand.

FIG. 8 is an illustration of a nanopore sensor/reader (junction potential type device) that can be used to sequence ssDNA that has been linearized by bound RPAs and/or SSBs. The nanopore sensor/reader measures current through each individual nucleotide as they pass through the electrode or conductor junction or gap.

In certain embodiments, a single-stranded nucleic acid having SSBs or RPAs bound on both side of a nanopore sensor or reader (SSBs or RPAs bound to first and second region of the single-stranded nucleic acid) can be sequenced. In certain embodiments, a single-stranded nucleic acid inserted into a nanopore sensor or reader is driven back and forth through a nanopore sensor or reader by current reversal (reversal of DC drive bias) such that the nucleic acid can be re-read each time it pass through the nanopore reader or flossed. FIG. 5 illustrates ssDNA with RPAs bound both cis and trans being flossed through a nanpore reader (e.g., alpha-hemolysin). In certain embodiments, a single-stranded nucleic acid having SSBs or RPAs bound on one side of a nanopore sensor or reader (bound to a first or a second region of the single-stranded nucleic acid) and a cap, enzyme or motor protein bound on the opposite side of the nanopore sensor or reader (bound to a second or a first region of the single-stranded nucleic acid) is driven back and forth through a nanopore sensor or reader by current reversal (reversal of DC drive bias) or by a motor protein (if present), such that the nucleic acid can be re-read each time it pass through the nanopore reader or flossed. In certain embodiments, driving a single-stranded nucleic acid back and forth through a nanopore sensor or reader is repeated multiple times. In certain embodiments, multiple times can be, but is not limited to, about 2 times to about 200 times, about 5 times to about 100 times, about 10 times to about 50 times, about 10 times to about 20 times or about 5, 6, 7, 8, 9, 10, 11, 12, 13, 14, 15, 16, 17, 18, 19, 20, 21, 22, 23, 24 or 25 times. In some embodiments, the number of times a single-stranded nucleic acid is passed back and forth through a nanopore sensor or reader is the number of times required to determine a consensus sequence for the molecule or a portion thereof.

In certain embodiments, a single-stranded nucleic acid comprising cap on one end of the molecule (on the cis or trans side of the nanopore) and SSBs or RPAs bound on the opposite side of the nanopore can be driven through the nanopore against the bound RPAs or SSBs such that the single-stranded nucleic acid is held taught, slowed, stretched or linearized, thus facilitating sequencing.

In certain embodiments, the sequencing can be targeted sequencing. In some embodiments, targeted sequencing comprises a cap bound to a single-stranded nucleic acid.

Nanopore Sensors or Readers, Single-Stranded Nucleic Acids and SSBs and/or RPAs

In certain embodiments, nanopore sensors and readers, single-stranded nucleic acids and SSBs and/or RPAs as described herein are provided together in an assemblage. In some embodiments, single-stranded nucleic acid is captured in a nanopore sensor or reader with SSB's and/or RPAs bound to one or more regions of the single-stranded nucleic acid. In some embodiments, a nanopore sensor or reader comprises a single-stranded nucleic acid, wherein a region of the single-stranded nucleic acid is on the cis side of a nanopore sensor or reader, a region of the single stranded nucleic acid is on the trans side of the nanopore sensor or reader and a region of the single-stranded nucleic acid is within the nanopore sensor or reader; the single-stranded nucleic acid comprises bound single-stranded binding proteins (SSBs) or replication protein A (RPAs) to a region on the cis side of the nanopore sensor or reader, to a region on the trans side of the nanopore sensor or reader or to a region on the cis side and a region on the trans side of the nanopore sensor or reader; and single-stranded binding proteins SSBs or RPAs are not bound to the single-stranded nucleic acid within the nanopore sensor or reader.

In certain embodiments, a cap or motor protein is also provided. In some embodiments, a nanopore sensor or reader comprises a single-stranded nucleic acid, wherein a region of the single-stranded nucleic acid is on the cis side of a nanopore sensor or reader, a region of the single stranded nucleic acid is on the trans side of the nanopore sensor or reader and a region of the single-stranded nucleic acid is within the nanopore sensor or reader; the single-stranded nucleic acid comprises a cap, motor protein or enzyme bound to a first region of the single-stranded nucleic acid located on the cis side of the nanopore sensor or reader and SSBs or RPAs bound to a second region of the single-stranded nucleic on the trans side of the nanopore sensor or reader or the single-stranded nucleic acid comprises single-stranded binding proteins (SSBs) or replication protein A (RPAs) bound to a region on the cis side of the nanopore sensor or reader and a cap, motor protein or enzyme bound to a second region of the single-stranded nucleic acid located on the trans side of the nanopore sensor or reader; and the SSBs or RPAs are not bound to the single-stranded nucleic acid within the nanopore sensor or reader.

In certain embodiments, also provided is an aqueous solution composed of a buffered electrolyte and/or an ionic solution. Non-limiting examples electrolytes that could be utilized include KCl, NaCl, LiCl, etc. buffered anywhere from pH 3.5 to 10.5 or within an unspecific usable range associated with the nanopore sensor or reader. In some embodiments, the electrolyte is at a concentration >0.3M, >0.5M, >1M, >1.5M, >2M, >2.5M, >3M, >3.5M, >4M, >4.5M, >5M, >5.5M or >6M. In some embodiments, the electrolyte is a salt specific to a halophile.

In certain embodiments, single-stranded DNA is inserted into a biological nanopore sensor or reader, RPA3s from Haloferax volcanii are bound to the single-stranded DNA, the electrolyte salt concentration is between about 3.0M to 4.0M and the temperature is less than or equal to 20° C. or about 5° C.

### REFERENCES

1. Shereda, R. D.; Kozlov, A. G.; Lohman, T. M.; Cox, M. M.; Keck, J. L., SSB as an organizer/mobilizer of genome maintenance complexes. Critical Reviews in Biochemistry and Molecular Biology 2008, 43 (5), 289-318.
2. Meyer, R. R.; Laine, P. S., The single-stranded DNA-binding protein of Escherichia coli. Microbiological reviews 1990, 54 (4), 342.
3. Hass, C. S.; Lam, K.; Wold, M. S., Repair-specific functions of replication protein A. Journal of Biological Chemistry 2012, 287 (6), 3908-3918.
4. Liu, T.; Huang, J., Replication protein A and more: single-stranded DNA-binding proteins in eukaryotic cells. Acta biochimica et biophysica Sinica 2016, 48 (7), 665-670.
5. Winter, J. A.; Patoli, B.; Bunting, K. A., DNA binding in high salt: analysing the salt dependence of replication protein A3 from the halophile haloferax volcanii. Archaea 2012, 2012.
6. Canganella, F.; Wiegel, J., Extremophiles: from abyssal to terrestrial ecosystems and possibly beyond. Naturwissenschaften 2011, 98 (4), 253-279.
7. Reed, C. J.; Lewis, H.; Trejo, E.; Winston, V.; Evilia, C., Protein adaptations in archaeal extremophiles. Archaea 2013, 2013.
8. Alkaliphiles. Taylor & Francis: 1999.
9. Rampelotto, P. H., Extremophiles and extreme environments. Multidisciplinary Digital Publishing Institute: 2013.
10. Ollivier, B.; Caumette, P.; Garcia, J.-L.; Mah, R., Anaerobic bacteria from hypersaline environments. Microbiological reviews 1994, 58 (1), 27-38.
11. Fendrihan, S.; Legat, A.; Pfaffenhuemer, M.; Gruber, C.; Weidler, G.; Gerbl, F.; Stan-Lotter, H., Extremely halophilic archaea and the issue of long-term microbial survival. Reviews in Environmental Science and Bio/technology 2006, 5 (2-3), 203-218.
12. Bakermans, C., Microbial Evolution under Extreme Conditions. De Gruyter: 2015.
13. Sharma, N.; Farooqi, M. S.; Chaturvedi, K. K.; Lal, S. B.; Grover, M.; Rai, A.; Pandey, P., The Halophile protein database. Database 2014, 2014.
14. Ali, I.; Prasongsuk, S.; Akbar, A.; Aslam, M.; Lotrakul, P.; Punnapayak, H.; Rakshit, S. K., Hypersaline habitats and halophilic microorganisms. Maejo International Journal of Science and Technology 2016, 10 (3), 330-345.

### EXAMPLES

The examples set forth below illustrate certain embodiments and do not limit the technology.

### Example 1: Haloferax volcanii Replication Protein A3 Coupled ssDNA Translocation of αHL

Haloferax volcanii replication protein A3 (RPA3) is a 14 kDa protein which has been shown to bind to ssDNA in high saline solutions making it an attractive target for use as an additive to modulate ssDNA translocation of alpha hemolysin (αHL) in conditions favorable to strand sequencing.⁵ The ability to use high molarity salt solutions in nanopore sequencing allows for sufficient signal to resolve differential current levels associated with individual bases (A, C, T, and G) at the low driving DC voltage and temperature required to maintain a reasonable translocation rate. Screening a variety of chemical additives as well as a modified single-stranded binding protein (SSB#2, A.acids #117-177 removed, Phe60-Trp) proved to be either incompatible with the experimental conditions, or of only marginal impact on the translocation rate and objective normalization of the individual events. The following chemical additives (proline, betaine, urea, spermidine, guanidine thiocyanate, trehalose and TOTO) were tested under high salt (greater than 1M) conditions and did not result in slowing of the translocation of ssDNA through a nonopore reader. The following SSBs/RPAs were tested for modulation of the translocation of ssDNA through a nanopore reader relative to free (no SSBs/RPAs) translocation and were found to not or only negligibly modulate translocation. In addition to SSB#2 (A.acids #117-177 removed, Phe60-Trp) (described above), E. coli SSBs #5 (remove 168-177, Aspl7, 42, 90, 95 to Arg), E. coli SSBs #6 (Asp17, 42, 90, 95, 170, 172, 173, 174 to Arg), T7g2.5 (Enterobacteria phage T7 single-stranded DNA binding protein gp2.5) and T4g32 (Enterobacteria phage T4 single-stranded DNA binding protein or helix-destabilizing protein). Human RPA (human replication protein A, 70 kDa DNA-binding subunit) produced very moderate slowing, ˜1.8-fold relative to free (no SSB) translocation The extremophile SSB/RPA TaqSSB (from Thermus aquaticus) exhibited ssDNA translocation through an αHL reader by more than 5-fold relative to free (no SSB) translocation.

Materials and Methods

Single-channel αHL recordings were made using the EBS AC/DC system and EBS Glass Nanopore Membranes (GNMs) with radii of 800-1000 nm, filled and bathed in 3.5 M NaCl, 10 mM Tris-HCl, 1 mM EDTA, pH 7.2. Temperature was maintained at a chosen setpoint by a thermoelectric cooler and a PID controller. Bilayers were formed by deposition of a minimal amount of 5 mg/ml DPhPC (Avanti) in n-Decane (Sigma-Aldrich) on the surface of the cis-side electrolyte bath followed by raising/lowering the cis-side solution level over the filled GNM aperture until resulting in reproducible seals measuring resistivity >300 GΩ and breakable by application of 1 V or a pore-specific measure of mechanical hydraulic pressure to the interior of the GNM.

Protein channels were isolated by adding 0.5 uL of EBS#238-1 YY 4S L135I αHL to the cis-side bath and applying sufficient mechanical hydraulic pressure to the interior of the GNM (usually ˜50% of the pore-specific pop pressure) and applying 800 ms pulses of escalating DC voltage followed by 200 ms rest periods at −120 mV to check for successful introduction of a protein pore. Pulses were in the range 120-360 mV with 30 mV steps every 5 seconds. After reaching 360 mV, the pulses remained at 360 mV until user action was taken or a protein insertion formed to stop the auto-insertion routine.

Haloferax volcanii RPA3 was provided by GenScript at a 1.2 mg/mL stock solution in 50 mM Tris-HCl, 150 mM NaCl, 10% glycerol, pH 8.0. Prior to use the stock preparation was buffer exchanged and concentrated into 3.5 M NaCl, 10 mM Tris-HCl, 1 mM EDTA, pH 7.2 by five 20 minute cycles of centrifugation at 14,000 rpm with a 10 kDa MWCO Millipore filtration unit. A volume between 30-35 uL was recovered from the centrifuge filter unit by spinning at 2000 rpm for 5 minutes. Concentrated RPA3 was combined with 5′-C40-ACT-C20-AGT-C20-ACT-C40-3′ ssDNA sequence at a 10:1 ratio of RPA3 to ssDNA and allowed to incubate benchtop for a minimum of 10 minutes prior to adding to the cis-side well of the EBS test cell.

Gene synthesis was performed for HvRPA3 and the subsequent gene was then cloned into an expression system/vector, along with a purification tag and cleavage site (located between the target gene and tag). After which, a strain of E. coli was transformed with the recombinant plasmid and subsequently cultured. The associated cells were then harvested and lysed, and the target protein (HvRPA3) was obtained via a two-step purification and utilized for ion channel recordings as described below. While a recombinant protein was used for the data presented below, native protein obtained directly from the organism of interest could also be utilized, in addition to various mutations thereof.

Results

RPA3 effects on translocation of the 5′-C40-ACT-C20-AGT-C20-ACT-C40-3′ sequence through the YY 4S L135I αHL channel in 3.5 M NaCl.

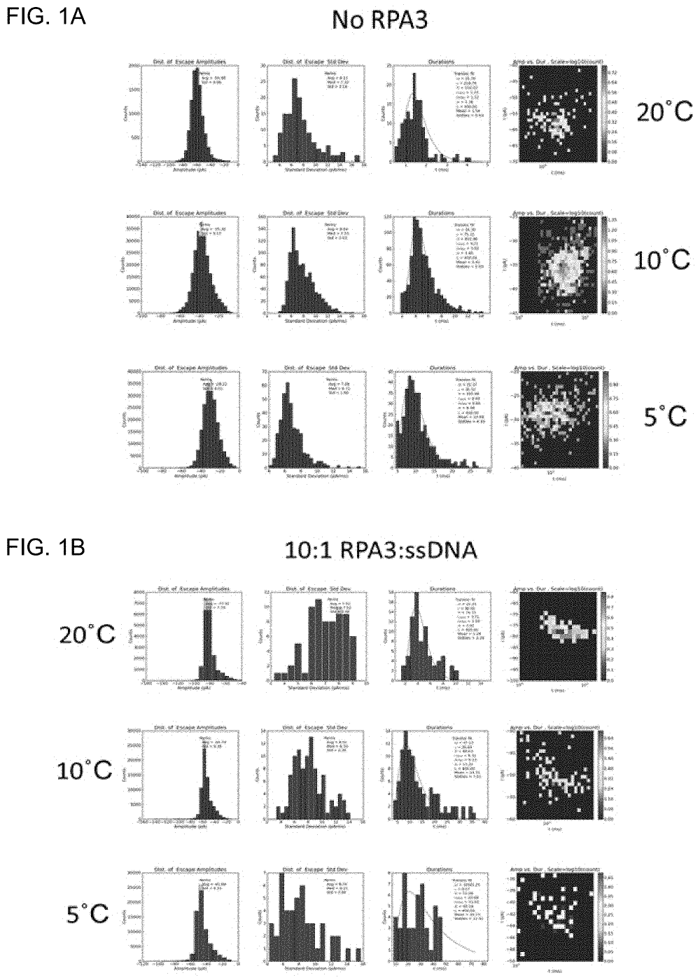

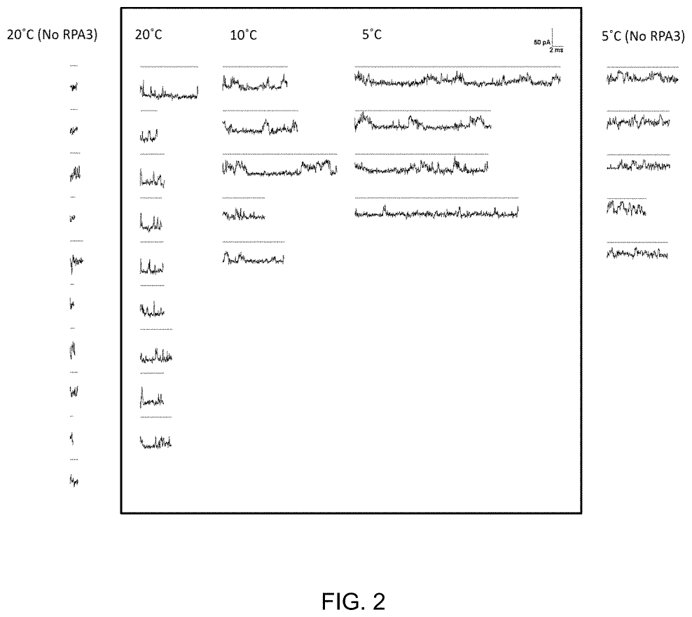

FIG. 2 shows select ACT-AGT-ACT sequence translocation event traces at −120 mV with and without 10:1 RPA3 additive. FIGS. 1A and 1B show distributions for the average amplitude standard deviation, and translocation duration of extracted ACT-AGT-ACT sequence translocation events of FIG. 2. Table 1(below) shows the measured statistics for the data depicted in FIGS. 1A, 1B and 2. Table 2 (below) shows the event rates and the residual current level in the absence and presence of RPA3.

At 20° C., the ACT-AGT-ACT sequence translocated at a rate reduced by roughly 50% with t-max=3.61 ms when RPA3 was added compared to RPA3-free translocation of the sequence. Further, the average residual current for the translocating sequence with RPA3 present (0.27) is 38% greater than when RPA3 is not present (0.19), possibly the result of an extended polymer structure due to restriction provide by bound RPA3.

TABLE 1
Measured Statistics for ACT-AGT-ACT
Translocation with 10:1 RPA3 Additive

| | Temp., | t-max, | | | No, | |
|---|---|---|---|---|---|---|
| Additive | C. | ms | lo, pA | lb, pA | pArms | Nb, pArms |
| — | 20 | 1.33 | 292 | 56.88 | 1.33 | 7.32 |
| RPA3 | 20 | 3.71 | 290 | 77.92 | 2 | 7.52 |
| — | 10 | 4.21 | 210.35 | 35.3 | 1.9 | 7.53 |
| RPA3 | 10 | 8.32 | 211.1 | 50.79 | 1.9 | 8.33 |
| — | 5 | 8.49 | 180.3 | 28.22 | 1.92 | 6.71 |
| RPA3 | 5 | 20.68 | 183.24 | 41.64 | 2.03 | 8.21 |

TABLE 2
Calculated Statistics for ACT-AGT-ACT
Translocation with 10:1 RPA3 Additive

| | Temp., | Event Rate, | Event Rate, | |
|---|---|---|---|---|
| Additive | C. | event/min | event/sec/uM | Ib/Io |
| — | 20 | 12.45 | 2.59E−05 | 0.19 |
| RPA3 | 20 | 13.50 | 2.81E−05 | 0.27 |
| — | 10 | 44.29 | 9.23E−05 | 0.17 |
| RPA3 | 10 | 10.89 | 2.27E−05 | 0.24 |
| — | 5 | 22.81 | 4.75E−05 | 0.16 |
| RPA3 | 5 | 3.92 | 1.63E−05 | 0.23 |

Visual inspection of individual extracted events (FIG. 2) illustrates that the current traces representing translocation are characterized by a baseline level of I/Iₒ=0.27 with resistive impulses of as much as 10% of Iₒ, or 29 pA at −120 mV. Lowering the measurement temperature to 10° C. and 5° C. extends the −120 mV translocation time to 8.1 and 20.68 ms, respectively, and continued to present an I/Iₒ measure ˜40% greater than without RPA3 and a fraction of events with current traces directly indicative of the sequence structure.

RPA3 Effects on Translocation of Poly(C)100 Sequence Through Wild-Type αHL Channel in 3.0 M NaCl.

To illustrate that the effects of RPA3 were not αHL mutant or ssDNA sequence specific, translocation of poly(C)100 through the wild-type αHL pore was monitored at −120 mV, 20 deg C. with 10:1 RPA3 present and compared to translocation data without RPA3. The result was a greater than 5× increase of the peak translocation time from 0.21 to 1.37 ms and a substantial 66% increase in I/Iₒ from 0.09 to 0.15±0.01 (see FIG. 3A (free translocation (messy)) and FIG. 3B (HvRPA3 bound translocation (clean)). This behavior is consistent with the slowing motion and increased I/Iₒ for the poly(C) dominant 5′-C40-ACT-C20-AGT-C20-ACT-C40-3′ strand translocating the YY-4S-L135I mutant αHL. The strong influence of RPA3 on ssDNA translocation appears to be sequence and αHL pore variety independent and presents opportunity for using higher DC driving voltages approaching −200 mV to monitor translocation through a nanopore with greater force applied against a restrictive ssDNA binding additive while maintaining translocation rate of 100 us/base at maintainable temperatures.

### Example 2: Listing of Certain Embodiments

Provided hereafter is a listing of certain non-limiting examples of embodiments of the technology.

A1. A method for translocating a single-stranded nucleic acid through a nanopore sensor or reader comprising:
contacting a single-stranded nucleic acid with single-stranded binding proteins (SSBs) or replication protein A (RPAs) under binding conditions in which the SSBs or RPAs bind to the single-stranded nucleic acid to produce a single-stranded nucleic acid with bound SSBs or bound RPAs; and
contacting the single-stranded nucleic acid with bound SSBs or bound RPAs under the binding conditions with the exterior of a nanopore sensor or reader and electrophoretically inducing translocation of the single-stranded nucleic acid through the nanopore sensor or reader.

A1.1. The method of embodiment A1, wherein contacting a single-stranded nucleic acid with single-stranded binding proteins (SSBs) or replication protein A (RPAs) and contacting the single-stranded nucleic acid with bound SSBs or bound RPAs under the binding conditions with the exterior of a nanopore sensor or reader comprises single-stranded nucleic acid previously inserted in a nanopore sensor or reader.

A1.2. The method of embodiment A1, wherein the single-stranded nucleic acid with bound SSBs or bound RPAs contacted with the exterior of a nanopore sensor or reader comprises a first region of single-stranded nucleic acid outside of the nanopore sensor or reader.

A1.3. The method of embodiment A1, wherein electrophoretically inducing translocation of the single-stranded nucleic acid through the nanopore sensor or reader comprises translocation of a region of the single-stranded nucleic acid not bound by SSB's or RPAs and located within the nanopore sensor or reader.

A1.4. A method for translocating a single-stranded nucleic acid through a nanopore sensor or reader comprising:
contacting a single-stranded nucleic acid inserted in a nanopore sensor or reader with single-stranded binding proteins (SSBs) or replication protein A (RPAs) under binding conditions, thereby generating single-stranded nucleic acid with SSBs or RPAs bound to a first region of the single-stranded nucleic outside of the nanopore sensor or reader; and
electrophoretically inducing translocation of a region of the single-stranded nucleic acid not bound by the SSBs or the RPAs through the nanopore sensor or reader.

A2. The method of any one of embodiments A1 to A1.4, wherein single-stranded nucleic acid is DNA.

A3. The method of any one of embodiments A1 to A1.4, wherein single-stranded nucleic acid is RNA.

A4. The method of any one of embodiments A1 to A3, wherein the nanopore sensor or reader is a biological nanopore sensor or reader.

A4.1. The method of embodiment A4, wherein the biological nanopore sensor or reader is alpha-hemolysin (αHL), aerolysin, Mycobacterium smegmatis porin A (MspA), Escherichia coli CsgG, or outer membrane protein F (OmpF).

A5. The method of any one of embodiments A1 to A3, wherein the nanopore sensor or reader is a synthetic nanopore sensor or reader.

A5.1. The method of embodiment A5, wherein the synthetic nanopore sensor or reader comprises an aperture with a diameter that prevents the single-stranded binding proteins (SSBs) or replication protein A (RPAs) bound to single-stranded nucleic acid from entering the nanopore sensor or reader.

A5.2. The method of embodiment A5.1, wherein the diameter is about 0.2 nm to about 10 nm.

A6. The method of any one of embodiments A1 to A5.2, wherein translocation of single-stranded nucleic acid with bound SSBs or bound RPAs through a nanopore sensor or reader is slower relative to translocation of single-stranded nucleic acid without bound SSBs or bound RPAs through a nanopore sensor or reader and/or associated current as a function of time noise level as single-stranded nucleic acid with bound SSBs or bound RPAs translocates through a nanopore sensor or reader is reduced relative to associated current as a function of time noise level as single-stranded nucleic acid without bound SSBs or bound RPAs translocates through a nanopore sensor or reader.

A6.1. The method of any one of embodiments A1.4 to A5.2, wherein the translocation through the nanopore sensor or reader of the region of the single-stranded nucleic acid not bound by SSBs or RPAs and having SSBs or RPAs bound to the first region is slower relative to the translocation through the nanopore sensor or reader of the region of the single-stranded nucleic acid not bound by SSBs or RPAs and without SSBs or RPAs bound to the first region.

A6.2. The method of embodiment A6.1, wherein translocation of the region of the single-stranded nucleic acid not bound by SSBs or RPAs through the nanopore reader or sensor is at a rate of about 100 microseconds to about 10 milliseconds.

A6.3. The method of any one of embodiments A1.4 to A5.2, wherein associated current as a function of time noise level for translocation through the nanopore sensor or reader of the region of the single-stranded nucleic acid not bound by SSBs or RPAs and having SSBs or RPAs bound to the first region is reduced relative to associated current as a function of time noise level for translocation through the nanopore sensor or reader of the region of the single-stranded nucleic acid not bound by SSBs or RPAs and without SSBs or RPAs bound to the first region.

A7. The method of any one of embodiments A1 to A6.3, wherein SSBs or RPAs are contacted with single-stranded nucleic acid at a high concentration of SSBs or RPAs to single-stranded nucleic acid.

A7.1. The method of embodiment A7, wherein concentration of SSBs or RPAs to single-stranded nucleic acid is greater than or equal to 10:1.

A7.2. The method of embodiment A7, wherein concentration of SSBs or RPAs to single-stranded nucleic acid is greater than or equal to 100:1.

A8. The method of any one of embodiments A1 to A7.2, wherein SSBs or RPAs are from an extremophile.

A9. The method of embodiment A 8, wherein the extremophile lives in an environment that is high temperature, low temperature, high pH, low pH, high salt concentration, high metal concentration, high chemical concentration or combinations thereof.

A10. The method of embodiment A8, wherein the method comprises conditions in which SSBs or RPAs from the extremophile have the highest binding affinity for single-stranded nucleic acid.

A11. The method of embodiment A10, wherein conditions comprise high temperature, low temperature, high pH, low pH, high chemical concentration or combinations thereof.

A11.1. The method of embodiment A9, wherein the conditions comprise conditions of the environment in which the extremophile lives and which comprise high temperature, low temperature, high pH, low pH, high salt concentration, high metal concentration, high chemical concentration or combinations thereof.

A12. The method of embodiment A10, wherein conditions comprise high salt and/or temperature less than or equal to 10° C.

A12.1. The method of embodiment A10, wherein binding conditions comprise high salt concentration and temperature less than or equal to 20° C.

A13. The method of any one of embodiments embodiment A8 to A12.1, wherein an extremophile is a halophile.

A14. The method of any one of embodiments A1 to A8 and A10 to A13, wherein conditions are a salt concentration of >0.3M, >0.5M, >1M, >1.5M, >2M, >2.5M, >3M, >3.5M, >4M, >4.5M, >5M, >5.5M or >6M.

A15. The method of any one of embodiments A8 to A12, wherein an extremophile is a thermophile.

A16. The method of any one of embodiments A1 to A8 and A10 to A12, wherein conditions are a temperature above 32° C., below 32° C., below 10° C., below 5° C., below 00° C. or below −5° C.

A16.1. The method of embodiment A16, wherein the binding conditions comprise high temperature and the temperature is above 32° C. or the binding conditions comprise low temperature and the temperature is below 5° C., below 00° C. or below −5° C.

A17. The method of any one of embodiments A8 to A14, wherein an extremophile is Haloferax volcanii.

A18. The method of any one of embodiments, A8 to A14 and A17, wherein RPAs are from Haloferax volcanii.

A18.1. The method of embodiment A18, wherein a RPA is RPA3.

A19. The method of embodiment A18.1, wherein the binding conditions for binding single-stranded nucleic acid is a salt concentration between 3M and 4M.

A19.1. The method of embodiment A18.1, wherein conditions for RPA3 binding comprise a salt concentration between 3M and 4M.

A20. The method of embodiment A18.1, wherein conditions for RPA3 binding single-stranded nucleic acid comprise a salt concentration greater than 0.5 M.

A21. The method of embodiment A19 or A20, wherein the temperature is less than about 32° C.

A21.1. The method of embodiment A21, wherein the temperature is less than or equal to about 20° C.

A21.2. The method of embodiment A21, wherein the temperature is about 5° C.

A22. The method of any one of embodiments A1 to A21.2, wherein SSBs or RPAs are native proteins or a portion thereof.

A23. The method of any one of embodiments A1 to A21, wherein SSBs or RPAs are recombinant proteins.

A24. The method of any one of embodiments A1 to A23, wherein SSBs or RPAs are mutated, engineered, chemically modified, or is a mutant form.

A25. The method of any one of embodiments A1 to A18 and A22 to A24, wherein SSBs or RPAs comprise one or more subunits that are in one or more oligomerization or multimerization states.

A26. The method of embodiment A25, wherein SSBs or RPAs are single subunits or monomeric proteins.

A26.1. The method of any one of embodiments A1 to A24, wherein SSBs or RPAs comprise single subunits or monomeric proteins.

A27. The method of embodiment A25, wherein SSBs or RPAs have multiple subunits and are homodimers, homotrimers, homotetramers, heterodimers, heterotrimers or heterotetramers.

A28. The method of any one of embodiments A1 to A27, wherein the method of translocating a single-stranded nucleic acid through a nanopore sensor or reader is used in a sequencing process.

A28.1. The method of embodiment A28, wherein the sequencing process comprises determining the sequence of the single-stranded nucleic acid or a portion thereof.

A28.2. The method of embodiment A28.1, wherein determining the sequence of the single-stranded nucleic acid or a portion thereof with SSBs or RPAs bound to a first region of the single-stranded nucleic acid increases the inter-nucleotide resolution relative to the inter-nucleotide resolution for determining the sequence of the single-stranded nucleic acid without SSBs or RPAs bound to a first region of the single-stranded nucleic acid.

A29. The method of any one of embodiments A1 to A27, wherein conditions are adjusted to influence recording measurements of a nanopore sensor or reader.

A29.1. The method of embodiment A29, wherein the recording measurements are current as a function of time.

A29.2. The method of embodiment A29, wherein the recording measurements are multiplexed through multiple nanopore sensors or readers.

A30. The method of embodiment A29, wherein the recording measurements are sensitivity, translocation time, signal amplitude, signal noise, signal to noise ratio and/or temporal resolution.

A30.1. The method of embodiment A29, wherein the recording measurements comprise sequence dependent current signatures.

A30.2. The method of embodiment A29, wherein the recording measurements in the presence of SSBs or RPAs bound to the first region of the single-stranded nucleic acid comprise a lower bandwidth measurement relative to the bandwidth measurement for recording measurements in the absence of SSBs or RPAs bound to the first region of the single-stranded nucleic acid.

A30.3. The method of embodiment A29, wherein the conditions comprise temperature and/or salt concentration.

A31. The method of any one of embodiments A1 to A30.3, wherein SSBs or RPAs can prevent single-stranded nucleic acid crosslinking, minimize the formation of secondary structures and annealing events, stretch a strand against an applied driving force, and/or slow the associated nanopore translocation rate.

A32. The method of any one of embodiments A1 to A30.3, wherein SSBs or RPAs enable the use of higher DC driving voltages to monitor translocation of single-stranded nucleic acid through a nanopore sensor or reader.

A33. The method of embodiment A32, wherein the DC driving voltages can be up to about −250 mV.

A34. The method of any one of embodiments A1 to A33, wherein the effect of SSBs or RPAs on the translocation rate of single-stranded nucleic acid is sequence independent.

A34.1. The method of any one of embodiments A1 to A33, wherein the effect of SSBs or RPAs on the translocation rate of single-stranded nucleic acid is sequence dependent.

A35. The method of any one of embodiments A1 to A34.1, wherein SSBs or RPAs are on the cis side of a nanopore sensor or reader.

A36. The method of any one of embodiments A1 to A35, wherein the single-stranded nucleic acid is linearized when translocation is electrophoretically induced.

B1. A method for translocating a single-stranded nucleic acid back and forth through a nanopore sensor or reader comprising:
providing single-stranded binding proteins (SSBs) or replication protein A (RPAs) under binding conditions on the cis side and the trans side of the nanopore sensor or reader, whereby when the single-stranded nucleic acid contacts the SSBs or RPAs, the SSBs or RPAs bind to the single-stranded nucleic acid to produce a single-stranded nucleic acid with bound SSBs or bound RPAs;
electrophoretically driving the single-stranded nucleic acid from bulk solution into the nanopore sensor or reader under binding conditions; and when the single-stranded nucleic acid is within the nanopore sensor or reader, electrophoretically driving the single-stranded nucleic acid back and forth through the nanopore sensor or reader under binding conditions, whereby the single-stranded nucleic acid is re-read.

B1.1. The method of embodiment B1, wherein the single-stranded nucleic acid is inserted into the nanopore reader or sensor and single-stranded binding proteins (SSBs) or replication protein A (RPAs) on the cis side of the nanopore reader or sensor bind to a first region of the single-stranded nucleic acid, single-stranded binding proteins (SSBs) or replication protein A (RPAs) on the trans side of the nanopore reader or sensor bind to a second region of the single-stranded nucleic acid and a third region between the first region and the second region is not bound by SSBs or RPAs and within the nanopore sensor or reader.

B1.2. A method for translocating a single-stranded nucleic acid back and forth through a nanopore sensor or reader comprising:
contacting a single-stranded nucleic acid inserted in a nanopore sensor or reader with single-stranded binding proteins (SSBs) or replication protein A (RPAs) on the cis and trans sides of the nanopore sensor or reader under binding conditions, thereby generating single-stranded nucleic acid with SSBs or RPAs bound to a first region of the single-stranded nucleic on the cis side of the nanopore sensor or reader and single-stranded nucleic acid with SSBs or RPAs bound to a second region of the single-stranded nucleic on the trans side of the nanopore sensor or reader; and
electrophoretically driving a third region of the single-stranded nucleic acid within the nanopore sensor or reader and not bound by the SSBs or the RPAs back and forth through the nanopore sensor or reader, whereby the third region of the single-stranded nucleic acid is translocated through the nanopore sensor or reader multiple times.

B1.3. The method of embodiment B1.2, wherein each time the third region of the single-stranded nucleic acid is translocated through the nanopore sensor or reader, the sequence is read by the nanopore sensor or reader.

B2. The method of any one of embodiments B1 to B1.3, wherein single-stranded nucleic acid is DNA.

B3. The method of any one of embodiments B1 to B1.3, wherein single-stranded nucleic acid is RNA.

B4. The method of any one of embodiments B1 to B3, wherein the nanopore sensor or reader is a biological nanopore sensor or reader.

B4.1. The method of embodiment B4, wherein the biological nanopore sensor or reader is alpha-hemolysin (αHL), aerolysin, Mycobacterium smegmatis porin A (MspA), Escherichia coli CsgG, or outer membrane protein F (OmpF).

B5. The method of any one of embodiments B1 to B3, wherein the nanopore sensor or reader is a synthetic nanopore sensor or reader.

B5.1. The method of embodiment B5, wherein the synthetic nanopore sensor or reader comprises an aperture with a diameter that prevents the single-stranded binding proteins (SSBs) or replication protein A (RPAs) bound to single-stranded nucleic acid from entering the nanopore sensor or reader.

B5.2. The method of embodiment B5.1, wherein the diameter is about 0.2 nm to about 10 nm.

B6. The method of any one of embodiments B1 to B5, wherein translocation of single-stranded nucleic acid with bound SSBs or bound RPAs through a nanopore sensor or reader is slower relative to translocation of single-stranded nucleic acid without bound SSBs or bound RPAs through a nanopore sensor or reader and/or associated current as a function of time noise level as single-stranded nucleic acid with bound SSBs or bound RPAs translocates through a nanopore sensor or reader is reduced relative to associated current as a function of time noise level as single-stranded nucleic acid without bound SSBs or bound RPAs translocates through a nanopore sensor or reader.

B6.1. The method of any one of embodiments B1.2 to B5.2, wherein the translocation through the nanopore sensor or reader of the third region of the single-stranded nucleic acid, with SSBs or RPAs bound to the first region and bound to the second region is slower relative to the translocation through the nanopore sensor or reader of the third region of the single-stranded nucleic acid without SSBs or RPAs bound to the first region and the second region.

B6.2. The method of embodiment B6.1, wherein translocation of the third region of the single-stranded nucleic acid through the nanopore reader or sensor is at a rate of about 100 microseconds to about 10 milliseconds.

B6.3. The method of any one of embodiments B1.2 to B5.2, wherein associated current as a function of time noise level for translocation through the nanopore sensor or reader of the third region of the single-stranded nucleic acid with SSBs or RPAs bound to the first region and the second region is reduced relative to associated current as a function of time noise level for translocation through the nanopore sensor or reader of the third region of the single-stranded nucleic acid without SSBs or RPAs bound to the first region and the second region.

B7. The method of any one of embodiments B1 to B6.3, wherein SSBs or RPAs are contacted with single-stranded nucleic acid at a high concentration of SSBs or RPAs to single-stranded nucleic acid.

B7.1. The method of embodiment B7, wherein concentration of SSBs or RPAs to single-stranded nucleic acid is greater than or equal to 10:1.

B7.2. The method of embodiment B7, wherein concentration of SSBs or RPAs to single-stranded nucleic acid is greater than or equal to 100:1.

B8. The method of any one of embodiments B1 to B7.2, wherein SSBs or RPAs are from an extremophile.

B9. The method of embodiment B8, wherein the extremophile lives in an environment that is high temperature, low temperature, high pH, low pH, high salt concentration, high metal concentration, high chemical concentration or combinations thereof.

B10. The method of embodiment B8, wherein the method is carried out under conditions in which SSBs or RPAs from the extremophile have the highest binding affinity for single-stranded nucleic acid.

B11. The method of embodiment B10, wherein conditions are high temperature, low temperature, high pH, low pH, high chemical concentration or combinations thereof.

B11.1. The method of embodiment B9, wherein the conditions comprise conditions of the environment in which the extremophile lives and which comprise high temperature, low temperature, high pH, low pH, high salt concentration, high metal concentration, high chemical concentration or combinations thereof.

B12. The method of embodiment B10, wherein conditions are high salt concentration and/or temperature less than or equal to 10° C.

B12.1. The method of embodiment B10, wherein binding conditions comprise high salt concentration and temperature less than or equal to 20° C.

B13. The method of any one of embodiments B8 to B12.1, wherein an extremophile is a halophile.

B14. The method of any one of embodiments B1 to B8 and B10 to B13, wherein conditions are a salt concentration >0.3M, >0.5M, >1M, >1.5M, >2M, >2.5M, >3M, >3.5M, >4M, >4.5M, >5M, >5.5M or >6M.

B15. The method of any one of embodiments B8 to B12, wherein an extremophile is a thermophile.

B16. The method of any one of embodiments B1 to B8 to B10 to B12.1, wherein conditions are a temperature above 32° C., below 32° C., below 10° C., below 5° C., below 00° C. or below −5° C.

B16.1. The method of embodiment B16, wherein the conditions comprise high temperature and the temperature is above 32° C. or the conditions comprise low temperature and the temperature is below 5° C., below 00° C. or below −5° C.

B17. The method of embodiment B8 to B14, wherein an extremophile is Haloferax volcanii.

B18. The method of any one of embodiments B8 to B14 and B17, wherein RPAs are from Haloferax volcanii.

B18.1. The method of embodiment B18, wherein a RPA is RPA3.

B19. The method of embodiment B18.1, wherein conditions for binding single-stranded nucleic acid is a salt concentration between 3M and 4M.

B19.1. The method of embodiment B18.1, wherein conditions for RPA3 binding comprise a salt concentration between 3M and 4M.

B20. The method of embodiment B18.1, wherein conditions for binding single-stranded nucleic acid is a salt concentration greater than 0.5 M.

B21. The method of embodiment B19 or B20, wherein temperature is less than about 32° C.

B21.1. The method of embodiment B21, wherein the temperature is less than or equal to about 20° C.

B21.2. The method of embodiment B21.1, wherein the temperature is about 5° C.

B22. The method of any one of embodiments B1 to B21.2, wherein SSBs or RPAs are native proteins or a portion thereof.

B23. The method of any one of embodiments B1 to B21.2, wherein SSBs or RPAs are recombinant proteins.

B24. The method of any one of embodiments B1 to B23, wherein SSBs or RPAs are a mutated, engineered, chemically modified, or a mutant form.

B25. The method of any one of embodiments B1 to B18 and B22 to B24, wherein SSBs or RPAs comprise one or more subunits that are in one or more oligomerization or multimerization states.

B26. The method of embodiment B25, wherein SSBs or RPAs are single subunits or monomeric proteins.

B26.1. The method of any one of embodiments B1 to B24, wherein SSBs or RPAs comprise single subunits or monomeric proteins.

B27. The method of embodiment B25, wherein SSBs or RPAs have multiple subunits and are homodimers, homotrimers, homotetramers, heterodimers, heterotrimers or heterotetramers.

B28. The method of any one of embodiments B1 to B27, wherein the method of translocating a single-stranded nucleic acid back and forth through a nanopore sensor or reader is used in a sequencing process.

B28.1. The method of embodiment B28, wherein the sequencing process comprises determining the sequence of the single-stranded nucleic acid or a portion thereof.

B28.2. The method of embodiment B28.1, wherein determining the sequence of the single-stranded nucleic acid or a portion thereof with SSBs or RPAs bound to a first region and a second region of the single-stranded nucleic acid increases the inter-nucleotide resolution relative to the inter-nucleotide resolution for determining the sequence of the single-stranded nucleic acid without SSBs or RPAs bound to a first region and the second region of the single-stranded nucleic acid.

B29. The method of any one of embodiments B1 to B27, wherein conditions are adjusted to influence recording measurements of a nanopore sensor or reader.

B29.1. The method of embodiment B29, wherein the recording measurements are current as a function of time.

B29.2. The method of embodiment B29, wherein the recording measurements are multiplexed through multiple nanopore sensors or readers.

B30. The method of embodiment B29, wherein the recording measurements are sensitivity, translocation time, signal amplitude, signal noise, signal to noise ratio and/or temporal resolution.

B30.1. The method of embodiment B29, wherein the recording measurements comprise sequence dependent current signatures.

B30.2. The method of embodiment B29, wherein the recording measurements in the presence of SSBs or RPAs bound to the first region and the second region of the single-stranded nucleic acid comprise a lower bandwidth measurement relative to the bandwidth measurement for recording measurements in the absence of SSBs or RPAs bound to the first region and the second region of the single-stranded nucleic acid.

B30.3. The method of embodiment B29, wherein the conditions comprise temperature and/or salt concentration.

B31. The method of any one of embodiments B1 to B30.3, wherein SSBs or RPAs can prevent single-stranded nucleic acid crosslinking, minimize the formation of secondary structures and annealing events, stretch the strand against an applied driving force, and/or slow the associated nanopore translocation rate.

B32. The method of any one of embodiments B1 to B30.3, wherein SSBs or RPAs enable the use of higher DC driving voltages to monitor translocation of single-stranded nucleic acid through a nanopore sensor or reader.

B33. The method of embodiment B32, wherein the DC driving voltages can be up to about −250 mV.

B34. The method of any one of embodiments B1 to B33, wherein the effect of SSBs or RPAs on the translocation rate of single-stranded nucleic acid is sequence independent.

B34.1. The method of any one of embodiments B1 to B33, wherein the effect of SSBs or RPAs on the translocation rate of single-stranded nucleic acid is sequence dependent.

B35. The method of any one of embodiments B1 to B34.1, wherein the single-stranded nucleic acid is linearized when electrophoretically driven back and forth through the nanopore sensor or reader.

C1. A method to linearize ssDNA or ssRNA within a nanopore sensor or reader, comprising;
capturing ssDNA or ssRNA within a nanopore sensor or reader to produce captured ssDNA or ssRNA;
contacting the captured ssDNA or ssRNA on the trans side of the nanopore sensor or reader with single-stranded binding proteins (SSBs) or replication protein A (RPAs) under binding conditions, wherein the SSBs or RPAs bind to a section of the ssDNA or ssRNA on the trans side to produce ssDNA or ssRNA with bound SSBs or bound RPAs; and
moving the ssDNA or ssRNA back out of the nanopore sensor or reader, whereby the ssDNA or ssRNA is linearized.

C2. The method of embodiment C1, wherein ssDNA or ssRNA is not bound by SSBs or RPAs on one side of a nanopore reader or sensor (cis) and ssDNA or ssRNA is bound by SSBs or RPAs on the other side of a nanopore sensor or reader (trans).

C2.1. The method of embodiment C1 or C2, wherein ssDNA or ssRNA has a cap, enzyme or motor protein on a strand or on the end of a strand.

C2.2. The method of embodiment C2.1, wherein the cap, enzyme or motor protein on a strand or on the end of a strand of ssDNA or ssRNA is on the cis side of the nanopore reader or sensor and the SSBs or RPAs on the ssDNA or ssRNA are on the trans side of the nanopore sensor or reader.

C2.3. A method to linearize ssDNA or ssRNA within a nanopore sensor or reader, comprising;
capturing ssDNA or ssRNA within a nanopore sensor or reader to produce captured ssDNA or ssRNA, wherein the captured ssDNA or ssRNA has a cap, enzyme or motor protein on a strand or on the end of a strand of the ssDNA or ssRNA on the trans side of the nanopore reader or sensor
contacting the captured ssDNA or ssRNA on the cis side of the nanopore sensor or reader with single-stranded binding proteins (SSBs) or replication protein A (RPAs) under binding conditions, wherein the SSBs or RPAs bind to a section of the ssDNA or ssRNA on the cis side to produce ssDNA or ssRNA with bound SSBs or bound RPAs; and
moving the ssDNA or ssRNA through the nanopore sensor or reader, whereby the ssDNA or ssRNA is linearized.

C2.4. The method of any one of embodiments C1 to C2.3, wherein the section of the captured ssDNA or ssRNA on the cis side of the nanopore sensor or reader comprises a first region of the ssDNA or ssRNA.

C2.4.1. The method of any one of embodiments C1 to C2.4, wherein the section of the captured ssDNA or ssRNA on the trans side of the nanopore sensor or reader comprises a second region of the ssDNA or ssRNA.

C2.4.2. The method of any one of embodiments C1 to C2.4.1, wherein the section of the captured ssDNA or ssRNA within a nanopore sensor or reader comprises a third region of the ssDNA or ssRNA that is not bound by single-stranded binding proteins (SSBs) or replication protein A (RPAs) or a cap, an enzyme or a motor protein.

C2.5. A method to linearize ssDNA or ssRNA within a nanopore sensor or reader, comprising;
contacting ssDNA or ssRNA inserted in a nanopore sensor or reader comprising a cap, motor protein or enzyme bound to a first region of the ssDNA or ssRNA located on the cis side of the nanopore sensor or reader with single-stranded binding proteins (SSBs) or replication protein A (RPAs) on the trans side of the nanopore sensor or reader under binding conditions, thereby generating ssDNA or ssRNA with SSBs or RPAs bound to a second region of the ssDNA or ssRNA on the trans side of the nanopore sensor or reader; or contacting ssDNA or ssRNA inserted in a nanopore sensor or reader comprising a cap, motor protein or enzyme bound to a second region of ssDNA or ssRNA located on the trans side of the nanopore sensor or reader, with single-stranded binding proteins (SSBs) or replication protein A (RPAs) on the cis side of the nanopore sensor or reader under binding conditions; thereby generating ssDNA or ssRNA with SSBs or RPAs bound to a first region of the single-stranded nucleic on the cis side of the nanopore sensor or reader; and
moving a third region of the ssDNA or ssRNA not bound by the SSBs, the RPAs, the cap, the motor protein or the enzyme through of the nanopore sensor or reader, whereby the ssDNA or ssRNA is linearized.

C2.6. A method for translocating ssDNA or ssRNA through a nanopore sensor or reader comprising:
contacting ssDNA or ssRNA inserted in a nanopore sensor or reader comprising a cap, motor protein or enzyme bound to a first region of the ssDNA or ssRNA located on the cis side of the nanopore sensor or reader with single-stranded binding proteins (SSBs) or replication protein A (RPAs) on the trans side of the nanopore sensor or reader under binding conditions; thereby generating ssDNA or ssRNA with SSBs or RPAs bound to a second region of the ssDNA or ssRNA on the trans side of the nanopore sensor or reader; or contacting ssDNA or ssRNA inserted in a nanopore sensor or reader comprising a cap, motor protein or enzyme bound to a second region of the ssDNA or ssRNA located on the trans side of the nanopore sensor or reader, with single-stranded binding proteins (SSBs) or replication protein A (RPAs) on the cis side of the nanopore sensor or reader under binding conditions, thereby generating ssDNA or ssRNA with SSBs or RPAs bound to a first region of the ssDNA or ssRNA on the cis side of the nanopore sensor or reader; and
driving a third region of the ssDNA or ssRNA not bound by the SSBs, the RPAs, the cap, the motor protein or the enzyme through the nanopore sensor or reader, whereby the third region of the ssDNA or ssRNA is translocated through the nanopore sensor or reader.

C2.6.1. The method of embodiment C2.6, wherein translocation of the third region of a single-stranded nucleic acid having SSBs or RPAs bound to a first region of the single stranded nucleic acid or having SSBs or RPAs bound to a second region of the single-stranded nucleic acid is slower relative to translocation of the third region not having SSBs or RPAs bound to a first region of the single stranded nucleic acid or not having SSBs or RPAs bound to a second region of the single-stranded nucleic acid.

C2.6.2. The method of embodiment C2.6.1, wherein translocation of the third region of the single-stranded nucleic acid through the nanopore reader or sensor is at a rate of about 100 microseconds to about 10 milliseconds.

C2.7. The method of any one of embodiments C2.6 to C2.6.2, wherein the third region of the ssDNA or ssRNA is translocated back and forth through the nanopore sensor or reader multiple times.

C2.8. The method of embodiment C2.7, wherein each time the third region of the ssDNA or ssRNA is translocated through the nanopore sensor or reader, the sequence is read by the nanopore sensor or reader.

C2.9. The method of embodiment C2.8, wherein the sequence of the ssDNA or ssRNA that is read by the nanopore sensor or reader is a targeted sequence determined by the position at which a cap is bound to the ssDNA or ssRNA.

C2.9.1. The method of any one of embodiments C1 to C2.9, wherein the ssDNA or ssRNA is held taught or stretched.

C2.9.2. The method of any one of embodiments C2.1 to C2.9.1, wherein ssDNA or ssRNA has a cap on the 3′ or 5 end and the cap is biotin/streptavidin, a hairpin or a g-quadreplex protein.

C2.9.3. The method of any one of embodiments C2.1 to C2.9.1, wherein ssDNA or ssRNA has an enzyme or motor protein bound to a strand and the enzyme or motor protein is an enzyme or motor protein of an extremophile, a halophile or thermophile.

C3. The method of any one of embodiments C1 to C2.9.3, wherein SSBs or RPAs are from a halophile and/or thermophile.

C4. The method of any one of embodiments C1 to C3, wherein moving ssDNA or ssRNA through a nanopore sensor or reader is by an applied force or by an enzyme or a motor protein.

C4.1. The method of embodiment C4, wherein the directionality of moving the ssDNA or ssRNA through a nanopore sensor or reader is determined by whether a cap, an enzyme or a motor protein is bound to the 3′ or 5′ end of the ssDNA or ssRNA.

C5. The method of embodiment C4, wherein ssDNA or ssRNA is moved through a nanopore sensor or reader by an applied force and the applied force is a DC bias that electrophoretically controls translocation of ssDNA or ssRNA.

C6. The method of embodiment C4, wherein ssDNA or ssRNA is moved through a nanopore sensor or reader by an enzyme.

C7. The method of embodiment C6, wherein an enzyme is a polymerase from an extremophile, a halophile or thermophile.

C7.1. The method of embodiment C4, wherein ssDNA or ssRNA is moved through a nanopore sensor or reader by a motor protein.

C7.2. The method of embodiment C7.1, wherein the motor protein is from an extremophile, a halophile or thermophile.

C8. The method of any one of embodiments C2.1 to C4 and C6 to C7.2, wherein an enzyme or motor protein function at high salt concentrations and/or low temperatures.

C8.1. The method of any one of embodiments C2.1 to C4 and C6 to C7.2, wherein the enzyme or motor protein function at high salt concentrations and/or low temperatures or high temperatures.

C9. The method of any one of embodiments C1 to C8.1, wherein the method is used in a sequencing application.

C10. The method of any one of embodiments C1 to C9, wherein a nanopore sensor or reader is a biological nanopore sensor or reader.

C10.1. The method of embodiment C10, wherein a biological nanopore sensor or reader is alpha-hemolysin (αHL), aerolysin, Mycobacterium smegmatis porin A (MspA), Escherichia coli CsgG, or outer membrane protein F (OmpF).

C11. The method of any one of embodiments C1 to C9, wherein a nanopore sensor or reader is a synthetic nanopore sensor or reader.

C11.1. The method of embodiment C11, wherein the synthetic nanopore sensor or reader comprises an aperture with a diameter that prevents the single-stranded binding proteins (SSBs) or replication protein A (RPAs) bound to ssDNA or ssRNA from entering the nanopore sensor or reader.

C11.2. The method of embodiment C11.1, wherein the diameter is about 0.2 nm to about 10 nm.

C12. The method of any one of embodiments C1 to C11.2, wherein SSBs or RPAs are contacted with ssDNA or ssRNA at a high concentration of SSBs or RPAs to ssDNA or ssRNA.

C12.1. The method of embodiment C12, wherein concentration of SSBs or RPAs to ssDNA or ssRNA is greater than or equal to 10:1.

C12.2. The method of embodiment C12, wherein concentration of SSBs or RPAs to ssDNA or ssRNA is greater than or equal to 100:1.

C13. The method of any one of embodiments C1 to C12.2, wherein SSBs or RPAs are from an extremophile.

C14. The method of embodiment C13, wherein an extremophile lives in an environment that is high temperature, low temperature, high pH, low pH, high salt concentration, high metal concentration, high chemical concentration or combinations thereof.

C15. The method of embodiment C13, wherein the method is carried out under conditions in which SSBs or RPAs from an extremophile have the highest binding affinity for ssDNA or ssRNA.

C16. The method of embodiment C15, wherein conditions are high temperature, low temperature, high pH, low pH, high chemical concentration or combinations thereof.

C16.1. The method of embodiment C14, wherein the conditions comprise conditions of the environment in which the extremophile lives and which comprise high temperature, low temperature, high pH, low pH, high salt concentration, high metal concentration, high chemical concentration or combinations thereof.

C17. The method of embodiment C15, wherein conditions are high salt and/or temperature less than or equal to 10° C.

C17.1. The method of embodiment C15, wherein binding conditions comprise high salt concentration and temperature less than or equal to 20° C.

C18. The method of any one of embodiments C13 to C17.1, wherein an extremophile is a halophile.

C19. The method of any one of embodiments C1 to C13 and C16.1 to C18, wherein conditions are a salt concentration >0.3M, >0.5M, >1M, >1.5M, >2M, >2.5M, >3M, >3.5M, >4M, >4.5M, >5M, >5.5M or >6M.

C20. The method of any one of embodiments C13 to C17, wherein an extremophile is a thermophile.

C21. The method of any one of embodiments C1 to C13, C15, C16, C16.1 and C20, wherein conditions are a temperature above 32° C., below 32° C., below 10° C., below 5° C., below 00° C. or below 5° C.

C21.1. The method of embodiment C21, wherein the conditions comprise high temperature and the temperature is above 32° C. or the conditions comprise low temperature and the temperature is below 5° C., below 00° C. or below −5° C.

C22. The method of any one of embodiments C13 to C18, wherein an extremophile is Haloferax volcanii.

C23. The method of any one of embodiments C13 to C18, wherein RPAs are from Haloferax volcanii.

C24. The method of embodiment C23, wherein a RPA is RPA3.

C25. The method of embodiment C24, wherein conditions for binding ssDNA or ssRNA is a salt concentration between 3M and 4M.

C25.1. The method of embodiment C25, wherein conditions for RPA3 binding comprise a salt concentration between 3M and 4M.

C26. The method of embodiment C24, wherein conditions for binding ssDNA or ssRNA is a salt concentration greater than 0.5 M.

C27. The method of any one of embodiments C25 to C26, wherein temperature is less than about 32° C.

C27.1. The method of embodiment C27, wherein the temperature is less than or equal to about 20° C.

C27.2. The method of embodiment C27.1, wherein the temperature is about 5° C.

C28. The method of any one of embodiments C1 to C27.2, wherein SSBs or RPAs are native proteins or a portion thereof.

C29. The method of any one of embodiments C1 to C27.2, wherein SSBs or RPAs are recombinant proteins.

C30. The method of any one of embodiments C1 to C29, wherein SSBs or RPAs are a mutated, engineered, chemically modified, or is a mutant form.

C31. The method of any one of embodiments C1 to C21.1 and C28 to C30, wherein SSBs or RPAs comprise one or more subunits that are in one or more oligomerization or multimerization states.

C32. The method of embodiment C31, wherein SSBs or RPAs are single subunits or monomeric proteins.

C32.1. The method of any one of embodiments C1 to C30, wherein SSBs or RPAs comprise single subunits or monomeric proteins.

C33. The method of embodiment C31, wherein SSBs or RPAs have multiple subunits and are homodimers, homotrimers, homotetramers, heterodimers, heterotrimers or heterotetramers.

C34. The method of any one of embodiments C1 to C33, wherein the method to linearize ssDNA or ssRNA is used in a sequencing process.

C34.1. The method of embodiment C34, wherein the sequencing process comprises determining the sequence of the ssDNA or ssRNA or a portion thereof.

C34.2. The method of embodiment C34.1, wherein determining the sequence of the ssDNA or ssRNA or a portion thereof with SSBs or RPAs bound to a first region or a second region of the ssDNA or ssRNA increases the inter-nucleotide resolution relative to the inter-nucleotide resolution for determining the sequence of the ssDNA or ssRNA without SSBs or RPAs bound to a first region or a second region of the ssDNA or ssRNA.

C35. The method of any one of embodiments C1 to C34.2, comprising obtaining recording measurements of the nanopore sensor or reader.

C36. The method of embodiment C35, wherein conditions are adjusted to influence recording measurements of a nanopore sensor or reader.

C37. The method of embodiment C35, wherein the recording measurements are current as a function of time.

C38. The method of embodiment C35, wherein the recording measurements are multiplexed through multiple nanopore sensors or readers.

C39. The method of embodiment C35, wherein the recording measurements are sensitivity, translocation time, signal amplitude, signal noise, signal to noise ratio and/or temporal resolution.

C40. The method of embodiment C35, wherein the recording measurements comprise sequence dependent current signatures.

C41. The method of embodiment 35, wherein the recording measurements in the presence of SSBs or RPAs bound to the first region or the second region of the ssDNA or ssRNA comprise a lower bandwidth measurement relative to the bandwidth measurement for recording measurements in the absence of SSBs or RPAs bound to the first region or the second region of the ssDNA or ssRNA.

C42. The method of embodiment C36, wherein the conditions comprise temperature and/or salt concentration.

C43. The method of any one of embodiments C1 to C42, wherein SSBs or RPAs can prevent single-stranded nucleic acid crosslinking, minimize the formation of secondary structures and annealing events, stretch the strand against an applied driving force, and/or slow the associated nanopore translocation rate.

C44. The method of any one of embodiments C1 to C42, wherein SSBs or RPAs enable the use of higher DC driving voltages to monitor translocation of single-stranded nucleic acid through a nanopore sensor or reader.

C45. The method of embodiment C44, wherein the DC driving voltages can be up to about −250 mV.

D1. A method for preparing single-stranded DNA or single-stranded RNA for translocation through a nanopore sensor or reader, comprising, separating the strands of double-stranded DNA or double-stranded RNA to produce single-stranded DNA or single-stranded RNA;
contacting the single-stranded DNA or single-stranded RNA with under binding conditions which the SSBs or RPAs bind to the single-stranded DNA or single-stranded RNA to produce single-stranded DNA or single-stranded RNA with bound SSBs or bound RPAs; and
contacting the single-stranded DNA or single-stranded RNA with bound SSBs or bound RPAs with a nanopore sensor or reader.

D2. The method of embodiment D1, wherein single-stranded DNA or single-stranded RNA with bound SSBs or bound RPAs is inhibited from hybridizing with itself or folding onto itself.

D2.1. The method of embodiment D1 or D2, wherein separating the strands of double-stranded DNA or double-stranded RNA is by chemical denaturation.

D3. The method of embodiment D2.1, wherein the chemical denaturation uses NaOH.

D4. The method of any one of embodiments D1 to D3, wherein a nanopore sensor or reader is a biological nanopore sensor or reader.

D4.1. The method of embodiment D4, wherein a biological nanopore sensor or reader is alpha-hemolysin (αHL), aerolysin, Mycobacterium smegmatis porin A (MspA), Escherichia coli CsgG, or outer membrane protein F (OmpF).

D5. The method of any one of embodiments D1 to D3, wherein a nanopore sensor or reader is a synthetic nanopore sensor or reader.

D6. The method of any one of embodiments D1 to D5, wherein SSBs or RPAs are contacted with single-stranded DNA or single-stranded RNA at a high concentration of SSBs or RPAs to single-stranded DNA or single-stranded RNA.

D7. The method of embodiment D6, wherein concentration of SSBs or RPAs to single-stranded DNA or single-stranded RNA is greater than or equal to 10:1.

D7.1. The method of embodiment D6, wherein concentration of SSBs or RPAs to single-stranded DNA or single-stranded RNA is greater than or equal to 100:1.

D8. The method of any one of embodiments D1 to D7.1, wherein SBBs or RPAs are from an extremophile.

D9. The method of embodiment D8, wherein an extremophile lives in an environment that is high temperature, low temperature, high pH, low pH, high salt concentration, high metal concentration, high chemical concentration or combinations thereof.

D10. The method of embodiment D8, wherein the method is carried out under conditions in which SSBs or RPAs from an extremophile have the highest binding affinity for single-stranded DNA or single-stranded RNA.

D11. The method of embodiment D10, wherein conditions are high temperature, low temperature, high pH, low pH, high chemical concentration or combinations thereof.

D12. The method of embodiment D10, wherein conditions are high salt and/or temperature less than or equal to 10° C.

D13. The method of any one of embodiments D8 to D12, wherein an extremophile is a halophile.

D14. The method of any one of embodiments D1 to D8 and D10 to D13, wherein conditions are a salt concentration >0.3M, >0.5M, >1M, >1.5M, >2M, >2.5M, >3M, >3.5M, >4M, >4.5M, >5M, >5.5M or >6M.

D15. The method of any one of embodiments D8 to D12, wherein an extremophile is a thermophile.

D16. The method of any one of embodiments D1 to D8 and D10 to D12, wherein conditions are a temperature above 32° C., below 32° C., below 10° C., below 5° C., below 00° C. or below −5° C.

D17. The method of any one of embodiments D8 to D14, wherein an extremophile is Haloferax volcanii.

D18. The method of any one of embodiments D8 to D14 and D17, wherein RPAs are from Haloferax volcanii.

D18.1. The method of embodiment D18, wherein a RPA is RPA3.

D19. The method of embodiment D18.1, wherein conditions for binding single-stranded DNA or single-stranded RNA is a salt concentration between 3M and 4M.

D20. The method of embodiment D18.1, wherein conditions for binding single-stranded DNA or single-stranded RNA is a salt concentration greater than 0.5 M.

D21. The method of embodiment D19 or D20, wherein temperature is less than about 32° C.

D22. The method of any one of embodiments D1 to D21, wherein SSBs or RPAs are native proteins or a portion thereof.

D23. The method of any one of embodiments D1 to D21, wherein SSBs or RPAs are recombinant proteins.

D24. The method of any one of embodiments D1 to D23, wherein SSBs or RPAs are a mutated, engineered, chemically modified, or is a mutant form.

D25. The method of any one of embodiments D1 to D18 and D22 to D24, wherein SSBs or RPAs comprise one or more subunits that are in one or more oligomerization or multimerization states.

D26. The method of any one of embodiments D1 to D24, wherein SSBs or RPAs are single subunits or monomeric proteins.

D27. The method of embodiment D25, wherein SSBs or RPAs have multiple subunits and are homodimers, homotrimers, homotetramers, heterodimers, heterotrimers or heterotetramers.

D28. The method of any one of embodiments D1 to D27, wherein the method is used in a sequencing process.

D29. The method of any one of embodiments D1 to D28, wherein SSBs or RPAs can prevent single-stranded DNA or single-stranded RNA crosslinking, minimize the formation of secondary structures and annealing events, stretch a strand against an applied driving force, and/or slow the associated nanopore translocation rate.

E1. A method for translocating single-stranded DNA through a nanopore sensor or reader comprising:
contacting the single-stranded DNA with RPA3s from Haloferax volcanii under binding conditions comprising a salt concentration greater than 0.5M to produce single-stranded DNA with bound RPA3s; and
contacting the single-stranded DNA with bound RPA3s under the binding conditions with the exterior of a nanopore sensor or reader and electrophoretically inducing translocation of the single-stranded DNA through the nanopore sensor or reader.

E1.1. The method of embodiment E1, wherein contacting single-stranded DNA with RPA3s from Haloferax volcanii and contacting the single-stranded DNA with bound RPA3s under the binding conditions with the exterior of a nanopore sensor or reader bind to the single-stranded DNA to produce a single-stranded DNA with bound RPA3s comprises single-stranded DNA previously inserted in a nanopore sensor or reader.

E1.2. The method of embodiment E1, wherein the single-stranded DNA with bound RPA3s contacted with the exterior of a nanopore sensor or reader comprises a first region of single-stranded DNA outside of the nanopore sensor or reader.

E1.3. The method of embodiment E1, wherein electrophoretically inducing translocation of the single-stranded DNA through the nanopore sensor or reader comprises translocation of a region of the single-stranded DNA not bound by RPA3s and located within the nanopore sensor or reader.

E1.4. A method for translocating a single-stranded DNA through a nanopore sensor or reader comprising:
contacting single-stranded DNA inserted in a nanopore sensor or reader with RPA3s from Haloferax volcanii under binding conditions comprising a salt concentration greater than 0.5M; thereby generating single-stranded DNA with RPA3s bound to a first region of the single-stranded DNA outside of the nanopore sensor or reader; and
electrophoretically inducing translocation of a region of the single-stranded DNA not bound by the RPA3s through the nanopore sensor or reader.

E2. The method of any one of embodiments E1 to E1.4, wherein binding conditions comprise temperatures below 32° C.

E3. The method of any one of embodiments E1 to E2, wherein a nanopore sensor or reader is a biological nanopore sensor or reader.

E4. The method of embodiment E3, wherein a biological nanopore sensor or reader is alpha-hemolysin (αHL), aerolysin, Mycobacterium smegmatis porin A (MspA), Escherichia coli CsgG, or outer membrane protein F (OmpF).

E5. The method of any one of embodiments E1 to E2, wherein a nanopore sensor or reader is a synthetic nanopore sensor or reader.

E5.1. The method of embodiment E5, wherein the synthetic nanopore sensor or reader comprises an aperture with a diameter that prevents the single-stranded binding proteins (SSBs) or replication protein A (RPAs) bound to single-stranded nucleic acid from entering the nanopore sensor or reader.

E5.2. The method of embodiment E5.1, wherein the diameter is about 0.2 nm to about 10 nm.

E6. The method of any one of embodiments E1 to E5.2, wherein translocation of single-stranded DNA with bound RPA3s through a nanopore sensor or reader is slower relative to translocation of single-stranded DNA without bound RPA3s through a nanopore sensor or reader and/or associated current as a function of time noise level as single-stranded DNA with bound RPA3s translocates through a nanopore sensor or reader is reduced relative to associated current as a function of time noise level as single-stranded DNA without bound RPA3s translocates through a nanopore sensor or reader.

E6.1. The method of any one of embodiments E1.4 to E5.2, wherein the translocation through the nanopore sensor or reader of the region of the single-stranded DNA not bound by RPA3s and having RPA3s bound to the first region is slower relative to the translocation through the nanopore sensor or reader of the region of the single-stranded DNA not bound by RPA3s and without RPA3s bound to the first region.

E6.2. The method of embodiment E6.1, wherein translocation of the region of the single-stranded DNA not bound by RPA3s through the nanopore reader or sensor is at a rate of about 100 microseconds to about 10 milliseconds.

E6.3. The method of any one of embodiments E1.4 to E5.2, wherein associated current as a function of time noise level for translocation through the nanopore sensor or reader of the region of the single-stranded DNA not bound by RPA3s and with RPA3s bound to the first region is reduced relative to associated current as a function of time noise level for translocation through the nanopore sensor or reader of the region of the single-stranded DNA not bound by RPA3s and without RPA3s bound to the first region.

E7. The method of any one of embodiments E1 to E6.3, wherein RPA3s are contacted with single-stranded DNA at a high concentration of RPA3s to single-stranded DNA.

E7.1. The method of embodiment E7, wherein concentration of RPA3s to single-stranded DNA is greater than or equal to 10:1.

E7.2. The method of embodiment E7, wherein concentration of RPA3s to single-stranded DNA is greater than or equal to 100:1.

E8. The method of any one of embodiments E1 to E7.2, wherein the method is carried out under conditions in which RPA3s have the highest binding affinity for single-stranded DNA.

E9. The method of embodiment E8, wherein conditions are high salt and/or temperature less than or equal to 10° C.

E9.1. The method of embodiment E8, wherein conditions comprise high salt concentration and temperature less than or equal to 20° C.

E10. The method of embodiment E9, wherein conditions are a salt concentration >0.3M, >0.5M, >1M, >1.5M, >2M, >2.5M, >3M, >3.5M, >4M, >4.5M, >5M, >5.5M or >6M.

E10.1. The method of embodiment E9.1, wherein conditions comprise a salt concentration >0.3M, >0.5M, >1M, >1.5M, >2M, >2.5M, >3M, >3.5M, >4M, >4.5M, >5M, >5.5M or >6M.

E11. The method of embodiment E9, wherein conditions for binding single-stranded DNA is a salt concentration between 3M and 4M.

E11.1. The method of embodiment E9. 1, wherein conditions for binding single-stranded DNA comprise a salt concentration between 3M and 4M.

E12. The method of embodiment E9, wherein conditions for binding single-stranded DNA is a salt concentration greater than 0.5 M.

E13. The method of embodiment Ell 1 or E12, wherein temperature is less than about 32° C.

E13.1. The method of embodiment E13, wherein temperature is less than or equal to about 20° C.

E13.2. The method of embodiment E13.1, wherein the temperature is about 5° C.

E14. The method of any one of embodiments E1 to E13.2, wherein RPA3s are native proteins.

E15. The method of any one of embodiments E1 to E13.2, wherein RPA3s are recombinant proteins.

E16. The method of any one of embodiments E1 to E15, wherein RPA3s are a mutated, engineered, chemically modified, or is a mutant form.

E17. The method of any one of embodiments E1 to E16, wherein the method of translocating a single-stranded DNA through a nanopore sensor or reader is used in a sequencing process.

E17.1. The method of embodiment E17, wherein the sequencing process comprises determining the sequence of the single-stranded DNA or a portion thereof.

E17.2. The method of embodiment E17.1, wherein determining the sequence of the single-stranded DNA or a portion thereof with RPA3s bound to a first region of the single-stranded DNA increases the inter-nucleotide resolution relative to the inter-nucleotide resolution for determining the sequence of the single-stranded DNA without RPA3s bound to a first region of the single-stranded DNA.

E18. The method of any one of embodiments E1 to E17.2, wherein conditions are adjusted to influence recording measurements of a nanopore sensor or reader.

E19. The method of embodiment E18, wherein the recording measurements are current as a function of time.

E20. The method of embodiment E18, wherein the recording measurements are multiplexed through multiple nanopore sensors or readers.

E21. The method of embodiment E18, wherein the recording measurements are sensitivity, translocation time, signal amplitude, signal noise, signal to noise ratio and/or temporal resolution.

E21.1. The method of embodiment E18, wherein the recording measurements comprise sequence dependent current signatures.

E21.2. The method of embodiment E18, wherein the recording measurements in the presence of RPA3s bound to the first region of the single-stranded DNA comprise a lower bandwidth measurement relative to the bandwidth measurement for recording measurements in the absence RPA3s bound to the first region of the single-stranded DNA.

E22. The method of any one of embodiments E1 to E21, wherein RPA3s can prevent ssDNA crosslinking, minimize the formation of secondary structures and annealing events, stretch a strand against an applied driving force, and/or slow the associated nanopore translocation rate.

E23. The method of any one of embodiments E1 to E22, wherein RPA3s enables the use of higher DC driving voltages to monitor translocation of single-stranded DNA through a nanopore sensor or reader.

E24. The method of embodiment E23, wherein the DC driving voltages can be up to about −250 mV.

E25. The method of any one of embodiments E1 to E24, wherein the effect of RPA3s on translocation rate of single-stranded DNA is sequence independent.

E26. The method of any one of embodiments E1 to E24, wherein the effect of RPA3s on translocation rate of the single-stranded DNA is sequence dependent.

E27. The method of any one of embodiments E1 to E26, wherein RPA3s are on the cis side of a nanopore sensor or reader.

E28. The method of any one of embodiments E1 to E27, wherein the single-stranded DNA is linearized when translocation is electrophoretically induced.

E29. A method for translocating a single-stranded DNA through a biological nanopore sensor or reader comprising:
contacting single-stranded DNA inserted in a biological nanopore sensor or reader with RPA3s from Haloferax volcanii under binding conditions comprising a salt concentration between about 3.0M to 4.0M and a temperature less than or equal to 20° C., thereby generating single-stranded DNA with RPA3s bound to a first region of the single-stranded DNA outside of the nanopore sensor or reader; and
electrophoretically inducing translocation of a region of the single-stranded DNA not bound by the RPA3s through the nanopore sensor or reader.

F1. A nanopore sensor or reader comprising:
a single-stranded nucleic acid, wherein a region of the single-stranded nucleic acid is on the cis side of a nanopore sensor or reader, a region of the single stranded nucleic acid is on the trans side of the nanopore sensor or reader and a region of the single-stranded nucleic acid is within the nanopore sensor or reader;
the single-stranded nucleic acid comprises bound single-stranded binding proteins (SSBs) or replication protein A (RPAs) to a region on the cis side of the nanopore sensor or reader, to a region on the trans side of the nanopore sensor or reader or to a region on the cis side and a region on the trans side of the nanopore sensor or reader; and
single-stranded binding proteins SSBs or RPAs are not bound to the single-stranded nucleic acid within the nanopore sensor or reader.

F2. The nanopore sensor or reader of embodiment F1, wherein the single-stranded nucleic acid is DNA.

F3. The nanopore sensor or reader of embodiment F1, wherein the single-stranded nucleic acid is RNA.

F4. The nanopore sensor or reader of any one of embodiments F1 to F3, wherein the nanopore sensor or reader is a biological nanopore sensor or reader.

F4.1. The nanopore sensor or reader of embodiment F4, wherein the biological nanopore sensor or reader is alpha-hemolysin (αHL), aerolysin, Mycobacterium smegmatis porin A (MspA), Escherichia coli CsgG, or outer membrane protein F (OmpF).

F5. The nanopore sensor or reader of any one of embodiments F1 to F3, wherein the nanopore sensor or reader is a synthetic nanopore sensor or reader.

F5.1. The nanopore sensor or reader of embodiment F5, wherein the synthetic nanopore sensor or reader comprises an aperture with a diameter that prevents the single-stranded binding proteins (SSBs) or replication protein A (RPAs) bound to single-stranded nucleic acid from entering the nanopore sensor or reader.

F5.2. The nanopore sensor or reader of embodiment F5.1, wherein the diameter is about 0.2 nm to about 10 nm.

F6. The nanopore sensor or reader of any one of embodiments F1 to F5.2, wherein SSBs or RPAs are from an extremophile.

F7. The nanopore sensor or reader of embodiment F6, wherein the SSBs or RPAs bind to single-stranded nucleic acid with high binding affinity under binding conditions comprising high temperature, low temperature, high pH, low pH, high salt concentration, high metal concentration, high chemical concentration or combinations thereof.

F8. The nanopore sensor or reader of embodiment F7, wherein the binding conditions comprise high salt concentration and a temperature less than or equal to 20° C.

F9. The nanopore sensor or reader of embodiment F6, wherein the extremophile is a halophile.

F10. The nanopore sensor or reader of embodiment F9, wherein the binding conditions for the halophile comprise a salt concentration >0.3M, >0.5M, >1M, >1.5M, >2M, >2.5M, >3M, >3.5M, >4M, >4.5M, >5M, >5.5M or >6M.

F11. The nanopore sensor or reader of embodiment F9, wherein the halophile is Haloferax volcanii.

F12. The nanopore sensor or reader of embodiment F11, wherein RPAs are from Haloferax volcanii.

F13. The nanopore sensor or reader of embodiment F11, wherein the RPAs are RPA3.

F14. The nanopore sensor or reader of embodiment F6, wherein the extremophile is a thermophile.

F15. The nanopore sensor or reader of embodiment F14, wherein the binding conditions for the thermophile comprise high temperature and the temperature is above 32° C. or the binding conditions comprise low temperature and the temperature is below 5° C., below 00° C. or below −5° C.

F16. The nanopore sensor or reader of any one of embodiments F1 to F15, wherein the SSBs or the RPAs are native proteins or a portion thereof.

F17. The nanopore sensor or reader of any one of embodiments F1 to F15, wherein the SSBs or the RPAs are recombinant proteins.

F18. The nanopore sensor or reader of any one of embodiments F1 to F15, wherein the SSBs or the RPAs are mutated, engineered, chemically modified, or is a mutant form.

F19. The nanopore sensor or reader of any one of embodiments F1 to F10 and F14 to F18, wherein the SSBs or the RPAs comprise one or more subunits that are in one or more oligomerization or multimerization states.

F20. The nanopore sensor or reader of any one of embodiments F1 to F18, wherein SSBs or RPAs are single subunits or monomeric proteins.

F21. The nanopore sensor or reader of embodiment F19, wherein the SSBs or the RPAs have multiple subunits and are homodimers, homotrimers, homotetramers, heterodimers, heterotrimers or heterotetramers.

F22. The nanopore sensor or reader of any one of embodiments F1 to F21, wherein the nanopore sensor or reader is part of a collection of nanopore sensors or readers for multiplexing.

F23. The nanopore sensor or reader of any one of claims F1 to F22, comprising a solution comprising an electrolyte at a concentration >0.3M, >0.5M, >1M, >1.5M, >2M, >2.5M, >3M, >3.5M, >4M, >4.5M, >5M, >5.5M or >6M, wherein the electrolyte is a salt specific to a halophile.

G1. A nanopore sensor or reader comprising:
a single-stranded nucleic acid, wherein a region of the single-stranded nucleic acid is on the cis side of a nanopore sensor or reader, a region of the single stranded nucleic acid is on the trans side of the nanopore sensor or reader and a region of the single-stranded nucleic acid is within the nanopore sensor or reader;
the single-stranded nucleic acid comprises a cap, motor protein or enzyme bound to a region of the single-stranded nucleic acid located on the cis side of the nanopore sensor or reader and SSBs or RPAs bound to a region of the single-stranded nucleic on the trans side of the nanopore sensor or reader or the single-stranded nucleic acid comprises single-stranded binding proteins (SSBs) or replication protein A (RPAs) bound to a region on the cis side of the nanopore sensor or reader and a cap, motor protein or enzyme bound to a region of the single-stranded nucleic acid located on the trans side of the nanopore sensor or reader; and
the SSBs or RPAs are not bound to the single-stranded nucleic acid within the nanopore sensor or reader.

G2. The nanopore sensor or reader of embodiment G1, wherein the single-stranded nucleic acid is DNA.

G3. The nanopore sensor or reader of embodiment G1, wherein the single-stranded nucleic acid is RNA.

G4. The nanopore sensor or reader of any one of embodiments G1 to G3, wherein the nanopore sensor or reader is a biological nanopore sensor or reader.

G4.1. The nanopore sensor or reader of embodiment G4, wherein the biological nanopore sensor or reader is alpha-hemolysin (αHL), aerolysin, Mycobacterium smegmatis porin A (MspA), Escherichia coli CsgG, or outer membrane protein F (OmpF).

G5. The nanopore sensor or reader of any one of embodiments G1 to G3, wherein the nanopore sensor or reader is a synthetic nanopore sensor or reader.

G5.1. The nanopore sensor or reader of embodiment G5, wherein the synthetic nanopore sensor or reader comprises an aperture with a diameter that prevents the single-stranded binding proteins (SSBs) or replication protein A (RPAs) bound to single-stranded nucleic acid from entering the nanopore sensor or reader.

G5.2. The nanopore sensor or reader of embodiment G5.1, wherein the diameter is about 0.2 nm to about 10 nm.

G6. The nanopore sensor or reader of any one of embodiments G1 to G5.2, wherein SSBs or RPAs are from an extremophile.

G7. The nanopore sensor or reader of embodiment G6, wherein the SSBs or RPAs bind to single-stranded nucleic acid with high binding affinity under binding conditions comprising high temperature, low temperature, high pH, low pH, high salt concentration, high metal concentration, high chemical concentration or combinations thereof.

G8. The nanopore sensor or reader of embodiment G7, wherein the binding conditions comprise high salt concentration and a temperature less than or equal to 20° C.

G9. The nanopore sensor or reader of embodiment G6, wherein the extremophile is a halophile.

G10. The nanopore sensor or reader of embodiment G9, wherein the binding conditions for the halophile comprise a salt concentration >0.3M, >0.5M, >1M, >1.5M, >2M, >2.5M, >3M, >3.5M, >4M, >4.5M, >5M, >5.5M or >6M.

G11. The nanopore sensor or reader of embodiment G9, wherein the halophile is Haloferax volcanii.

G12. The nanopore sensor or reader of embodiment G11, wherein RPAs are from Haloferax volcanii.

G13. The nanopore sensor or reader of embodiment G11, wherein the RPAs are RPA3.

G14. The nanopore sensor or reader of embodiment G6, wherein the extremophile is a thermophile.

G15. The nanopore sensor or reader of embodiment G14, wherein the binding conditions for the thermophile comprise high temperature and the temperature is above 32° C. or the binding conditions comprise low temperature and the temperature is below 5° C., below 00° C. or below −5° C.

G16. The nanopore sensor or reader of any one of embodiments G1 to G15, wherein the SSBs or the RPAs are native proteins or a portion thereof.

G17. The nanopore sensor or reader of any one of embodiments G1 to G15, wherein the SSBs or the RPAs are recombinant proteins.

G18. The nanopore sensor or reader of any one of embodiments G1 to G15, wherein the SSBs or the RPAs are mutated, engineered, chemically modified, or is a mutant form.

G19. The nanopore sensor or reader of any one of embodiments G1 to G10 and G14 to G18, wherein the SSBs or the RPAs comprise one or more subunits that are in one or more oligomerization or multimerization states.

G20. The nanopore sensor or reader of any one of embodiments G1 to G18, wherein SSBs or RPAs are single subunits or monomeric proteins.

G21. The nanopore sensor or reader of embodiment G19, wherein the SSBs or the RPAs have multiple subunits and are homodimers, homotrimers, homotetramers, heterodimers, heterotrimers or heterotetramers.

G22. The nanopore sensor or reader of any one of embodiments G1 to G21, wherein the single-stranded nucleic acid has a cap on the 3′ or 5 end and the cap is biotin/streptavidin, a hairpin or a g-quadreplex protein.

G22.1. The nanopore sensor or reader of embodiment G22, wherein the directionality of moving the single-stranded nucleic acid through the nanopore sensor or reader is determined by whether the cap is bound to the 3′ or 5′ end of the single-stranded nucleic acid.

G23. The nanopore sensor or reader of any one of embodiments G1 to G21, wherein the single-stranded nucleic acid has an enzyme or motor protein bound to a strand and the enzyme or motor protein is an enzyme or motor protein of an extremophile, a halophile or thermophile.

G23.1. The nanopore sensor or reader of embodiment G23, wherein the single-stranded nucleic acid has an enzyme bound to a strand the enzyme is a polymerase from an extremophile, a halophile or thermophile.

G24. The nanopore sensor or reader of embodiment G23 or G23.1, wherein the enzyme or motor protein function at high salt concentrations and/or low temperatures or high temperatures.

G25. The nanopore sensor or reader of any one of embodiments G1 to G24, wherein the nanopore sensor or reader is part of a collection of nanopore sensors or readers for multiplexing.

G26. The nanopore sensor or reader of any one of embodiments G1 to G25, comprising a solution comprising an electrolyte at a concentration >0.3M, >0.5M, >1M, >1.5M, >2M, >2.5M, >3M, >3.5M, >4M, >4.5M, >5M, >5.5M or >6M, wherein the electrolyte is a salt specific to a halophile.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A method for translocating a single-stranded nucleic acid through a nanopore sensor or reader comprising:
contacting a single-stranded nucleic acid inserted in a nanopore sensor or reader with single-stranded binding proteins (SSBs) or replication protein A (RPAs) under binding conditions, thereby generating single-stranded nucleic acid with SSBs or RPAs bound to a first region of the single-stranded nucleic outside of the nanopore sensor or reader; and
electrophoretically inducing translocation of a second region of the single-stranded nucleic acid not bound by the SSBs or the RPAs through the nanopore sensor or reader.

2. The method of claim 1, wherein single-stranded nucleic acid is DNA.

3. The method of claim 1, wherein single-stranded nucleic acid is RNA.

4. The method of claim 1, wherein the nanopore sensor or reader is a biological nanopore sensor or reader.

5. The method of claim 4, wherein the biological nanopore sensor or reader is alpha-hemolysin (αHL), aerolysin, Mycobacterium smegmatis porin A (MspA), Escherichia coli CsgG, or outer membrane protein F (OmpF).

6. The method of claim 1, wherein the nanopore sensor or reader is a synthetic nanopore sensor or reader.

7. The method of claim 1, wherein the translocation through the nanopore sensor or reader of the region of the single-stranded nucleic acid not bound by SSBs or RPAs and having SSBs or RPAs bound to the first region is slower relative to the translocation through the nanopore sensor or reader of the region of the single-stranded nucleic acid not bound by SSBs or RPAs and without SSBs or RPAs bound to the first region.

8. The method of claim 1, wherein SSBs or RPAs are contacted with single-stranded nucleic acid at a concentration of SSBs or RPAs to single-stranded nucleic acid of greater than or equal to 10:1.

9. The method of claim 1, wherein the SSBs or the RPAs are from an extremophile.

10. The method of claim 1, wherein conditions comprise high temperature, low temperature, high pH, low pH, high salt concentration, high chemical concentration or combinations thereof.

11. The method of claim 9, wherein the SSBs or RPAs are from an extremophile that is a halophile.

12. The method of claim 11, wherein the SSBs or RPAs from the halophile bind to single-stranded nucleic acid under conditions comprising high salt concentration and the salt concentration is >0.3M, >0.5M, >1M, >1.5M, >2M, >2.5M, >3M, >3.5M, >4M, >4.5M, >5M, >5.5M or >6M.

13. The method of claim 11, wherein the RPAs are RPA3 of Haloferax volcanii.

14. The method of claim 9, wherein the SSBs or RPAs are from an extremophile that is a thermophile.

15. The method of claim 14, wherein the SSBs or RPAs from the thermophile bind to single-stranded nucleic acid under conditions comprising high temperature and the temperature is above 32° C. or the conditions comprise low temperature and the temperature is below 5° C., below 00° C. or below −5° C.

16. The method of claim 1, comprising a sequencing process to determine the sequence of the single-stranded nucleic acid or portion thereof.

17. The method of claim 16, wherein determining the sequence of the single-stranded nucleic acid or a portion thereof with SSBs or RPAs bound to a first region of the single-stranded nucleic acid increases the inter-nucleotide resolution relative to the inter-nucleotide resolution for determining the sequence of the single-stranded nucleic acid without SSBs or RPAs bound to a first region of the single-stranded nucleic acid.

18. The method of claim 1, wherein the single-stranded nucleic acid is linearized when translocation is electrophoretically induced.

19. A method for translocating a single-stranded nucleic acid back and forth through a nanopore sensor or reader comprising:
contacting a single-stranded nucleic acid inserted in a nanopore sensor or reader with single-stranded binding proteins (SSBs) or replication protein A (RPAs) on the cis and trans sides of the nanopore sensor or reader under binding conditions, thereby generating single-stranded nucleic acid with SSBs or RPAs bound to a first region of the single-stranded nucleic on the cis side of the nanopore sensor or reader and single-stranded nucleic acid with SSBs or RPAs bound to a second region of the single-stranded nucleic on the trans side of the nanopore sensor or reader; and
electrophoretically driving a third region of the single-stranded nucleic acid within the nanopore sensor or reader and not bound by the SSBs or the RPAs back and forth through the nanopore sensor or reader, whereby the third region of the single-stranded nucleic acid is translocated through the nanopore sensor or reader multiple times.

20. A nanopore sensor or reader comprising:
a single-stranded nucleic acid, wherein a region of the single-stranded nucleic acid is on the cis side of a nanopore sensor or reader, a region of the single stranded nucleic acid is on the trans side of the nanopore sensor or reader and a region of the single-stranded nucleic acid is within the nanopore sensor or reader;
the single-stranded nucleic acid comprises bound single-stranded binding proteins (SSBs) or replication protein A (RPAs) to a region on the cis side of the nanopore sensor or reader, to a region on the trans side of the nanopore sensor or reader or to a region on the cis side and a region on the trans side of the nanopore sensor or reader; and
single-stranded binding proteins SSBs or RPAs are not bound to the single-stranded nucleic acid within the nanopore sensor or reader.
